(19) United States
(12) Patent Application Publication
(10) Pub. No.: US 2008/0312712 A1
(43) Pub. Date: Dec. 18, 2008

(54) Implantable Devices and Methods for Stimulation of Cardiac or Other Tissues

(75) Inventor: Abraham Penner, Tel Aviv (IL)

Correspondence Address: SUTHERLAND ASBILL & BRENNAN LLP, 999 PEACHTREE STREET, N.E., ATLANTA, GA, 30309, US

(73) Assignee: E-PACING, INC., Wilmington, DE (US)

(21) Appl. No.: 12/136,812

(22) Filed: Jun. 11, 2008

Related U.S. Application Data
(60) Provisional application No. 60/943,593, filed on Jun. 13, 2007.
(60) Provisional application No. 60/945,107, filed on Jun. 20, 2007.

(51) Int. Cl. A61N 1/05

(52) U.S. Cl. 607/40

(57) ABSTRACT

An implantable stimulation system is provided for stimulation of the heart, phrenic nerve, gastric system, or other tissue structures accessible via a patient's upper gastrointestinal system or airway. The stimulation system includes an implantable controller housing including a pulse generator; at least one electrical lead attachable to the pulse generator; and at least one electrode carried by the electrical lead that is positionable and fixable within the upper gastrointestinal tract or airway. The controller housing may be adaptable for subcutaneous implantation, or within the upper gastrointestinal tract or airway, wherein the controller housing is proportioned to substantially permit fluid and solid flow through the upper gastrointestinal tract or airway about the controller housing. The pulse generator may be operable to deliver one or more electrical pulses effective in cardiac pacing, cardiac defibrillation, cardioversion, cardiac resynchronization therapy, diaphragm pacing, phrenic nerve stimulation, gastric electrical stimulation, or a combination thereof.

### CROSS-REFERENCE TO RELATED APPLICATIONS

Priority is claimed to U.S. Provisional Application No. 60/943,593, filed Jun. 13, 2007, and to U.S. Provisional Application No. 60/945,107, filed Jun. 20, 2007, both of which are incorporated herein by reference.

### BACKGROUND OF THE INVENTION

This invention is generally in the field of implantable medical devices and treatment methods, and more particularly devices and methods for treating cardiac deficiencies with electrical stimulation.

Certain cardiac deficiencies, such as cardiac arrhythmias including bradycardia and tachycardia are typically treated by pacemakers or implantable cardioverter-defibrillators. A pacemaker is an electronic device that may pace or regulate the beating of a patient's heart by delivering precisely timed electrical stimulation to certain areas of the heart, depending upon the condition being treated. For example, bradycardia, where the heart rate is too slow, or tachycardia, where heart rate is too fast, may be treated by performing cardiac pacing. As used herein, the term “pacemaker” may refer to any cardiac rhythm management device that is operable to perform pacing functionality, regardless of any other functions it may perform.

Other cardiac stimulation devices may include implantable cardioverter-defibrillators, which may also be referred to herein as “cardioverters,”, “defibrillators,” or “ICD.” Implantable cardioverter-defibrillators perform functions similar to pacemakers by delivering electrical pulses, though they are most-often used to treat sudden cardiac arrhythmias such as atrial or ventricular fibrillation or ventricular tachycardia. Most ICDs operate by monitoring the rate and/or rhythm of the heart and deliver electrical pulses and/or electrical shocks when abnormalities are detected. For example, some ICDs may only deliver electrical shocks, while other ICDs may first deliver lower power electrical pulse to pace the heart prior to delivering electrical shocks.

In order to electrically stimulate the heart, electrodes are typically positioned and fixed close to the required stimulation site. In certain conventional cardiac stimulation techniques, a transvenous electrode is delivered by transvenous catheterization to the right atrium, the right ventricle, or both for performing dual chambers pacing. Other conventional cardiac stimulation devices include epicardial electrodes delivered to the epicardium at various locations.

In addition to generating and delivering electrical stimulation to a patient's heart, cardiac treatment devices often measure various physiological parameters to aid in detecting and treating cardiac deficiencies. For example, observing the heart's electrical activity allows for detecting many heart deficiencies, including, but not limited to, bradycardia, tachycardia, atrial fibrillation, and myocardial infraction. In addition, the synchronization may be detected between relative heart chambers, including the delay between right atrium and right ventricle (AV delay) and the delay between the right and left ventricles (V-V), which may assist in detecting and treating heart deficiencies. Furthermore, certain conventional cardiac devices measure electrical impedance around the heart to detect fluid congestion in the lungs, which may be indicative of congestive heart failure. Conventional cardiac devices may further include additional sensors, such as accelerometers, flow monitors, oxygen sensors, for example, for measuring other conditions related to a patient's cardiac performance.

Such conventional cardiac stimulation and sensing devices and techniques require a complex and highly invasive implantation procedures for electrode and pulse generator placement. Infections and other risks are associated with such highly invasive procedures. Electrical leads carrying the electrodes or other sensors are subjected to mechanical fatigue, as a result of the conventional delivery paths typically dictated by vasculature or cardiac anatomy, causing lead or electrode failure. It thus would be desirable to provide alternative systems, devices, and methods for positioning and fixing of stimulation electrodes proximate to desired stimulation sites, particularly for cardiac stimulation. It also would be desirable to provide systems, devices, and methods for minimally invasive or non-invasive implantation of a pulse generator to provide electrical stimulation signals through electrical leads to the stimulation electrodes.

### SUMMARY OF THE INVENTION

An implantable stimulation system is provided for stimulation of the heart, phrenic nerve, gastric system, or other tissue structures accessible via a patient's upper gastrointestinal system, airway, or a combination thereof. The stimulation system includes an implantable controller housing which includes a pulse generator; at least one electrical lead attachable to the pulse generator; and at least one electrode carried by the at least one electrical lead, wherein the at least one electrode positionable and fixable at a selected position within the patient's upper gastrointestinal tract or airway. The controller housing may be adaptable for subcutaneous implantation, or alternatively, at a selected position within the patient's upper gastrointestinal tract or airway, wherein the controller housing is proportioned to substantially permit fluid and solid flow through the upper gastrointestinal tract or airway about the controller housing. The pulse generator may be operable to deliver one or more electrical pulses effective in cardiac pacing, cardiac defibrillation, cardioversion, cardiac resynchronization therapy, diaphragm pacing, phrenic nerve stimulation, gastric electrical stimulation, or a combination thereof.

In one embodiment, the system may further include one or more cannulae adaptable for passage of the at least one electrical lead through a wall of the upper gastrointestinal tract or airway. In another embodiment, the system may further include one or more tissue interfaces for wirelessly communicating an electrical signal through a wall of the upper gastrointestinal tract or airway.

In another aspect, a method is provided for implanting a stimulation system in a patient in need thereof. The method may include implanting in the patient a controller housing including a pulse generator; and positioning at least one electrode, which is carried by at least one electrical lead (which is attached to the pulse generator) at a selected position within the upper gastrointestinal tract or airway of the patient. The method may include implanting the controller housing at a subcutaneous location within the patient or within the upper gastrointestinal tract or the airway of the patient.

In another aspect, a method is provided for implanting a stimulation system in a patient in need thereof. The method may include delivering a controller housing including a pulse generator through the patient's digestive tract; penetrating a wall of the esophagus of the digestive tract and forming an aperture therein; passing the controller housing through the aperture; and fixing the controller housing to an external surface of said wall of the esophagus. The method may further include delivering at least one electrode carried by at least one electrical lead attached to the pulse generator, through the digestive tract; passing the at least one electrode and the at least one electrical lead through the aperture; and fixing the at least one electrode to the epicardium of the patient's heart.

### BRIEF DESCRIPTION OF THE DRAWINGS

FIGS. 1A-1B illustrate a human gastrointestinal system and a human pulmonary system.

FIG. 2 is a diagram of a cardiac device placement according to one embodiment of the invention.

FIG. 3 is a diagram of a cardiac device placement according to one embodiment of the invention.

FIG. 4 is a diagram of a cardiac device placement according to one embodiment of the invention.

FIG. 5 is a diagram of a cardiac device placement according to one embodiment of the invention.

FIG. 6 is a diagram of a cardiac device placement according to one embodiment of the invention.

FIGS. 7A-7C are diagrams of electrical lead placement according to some embodiments of the invention.

FIGS. 8A-8F are diagrams of devices for anchoring a controller housing according to some embodiments of the invention.

FIG. 9 is a schematic diagram of a controller housing according to one embodiment of the invention.

FIG. 10 is a functional diagram of an electronic controller according to one embodiment of the invention.

FIGS. 11A-11B are diagrams of controller housings according to some embodiments of the invention.

FIGS. 12A-12G are diagrams of electrodes according to some embodiments of the invention.

FIGS. 13A-13D are diagrams of electrical leads according to some embodiments of the invention FIGS. 14A-14B are diagrams of cannulae according to some embodiments of the invention.

FIGS. 15A-15C are diagrams of a cannula implantable within an upper gastrointestinal tract and airway according to one embodiment of the invention.

FIGS. 16A-16B are diagrams of a cannula implantable within an upper gastrointestinal tract and airway according to one embodiment of the invention.

FIG. 17 is a diagram of a tissue interface according to one embodiment of the invention.

FIG. 18 is a flowchart of a method of implanting a cardiac device according to one embodiment of the invention.

FIG. 19 is a flowchart of a method of implanting an electrode according to one embodiment of the invention.

FIG. 20 is a flowchart of a method of implanting a controller housing according to one embodiment of the invention.

FIG. 21 is a flowchart of a method of testing an implanted electrode according to one embodiment of the invention.

FIG. 22 is a flowchart of a method of implanting a controller housing and an electrode according to one embodiment of the invention.

FIG. 23 is a diagram of a cardiac device placement according to one embodiment of the invention.

FIG. 24 is a diagram of a cardiac device placement according to one embodiment of the invention.

FIG. 25 is a diagram of a cardiac device placement according to one embodiment of the invention.

FIG. 26 is a flowchart of a method of implanting a cardiac device according to one embodiment of the invention.

FIG. 27 is a flowchart of a method of implanting a cardiac device according to one embodiment of the invention.

FIG. 28 is a flowchart of a method of implanting a cardiac device according to one embodiment of the invention.

FIG. 29 is a flowchart of a method of electrically stimulating a heart according to one embodiment of the invention.

### DETAILED DESCRIPTION OF THE INVENTION

Implantable medical devices and methods are provided for stimulation of cardiac or other tissues via electrodes implanted within a patient's upper gastrointestinal tract and, optionally, within a patient's airway. The human anatomy beneficially provides access to electrode implantation sites within the patient's upper gastrointestinal tract, such as the esophagus and the stomach, and the airway that are in close proximity to areas of the heart and other tissues, and thus allows for alternative implantation devices and methods for electrically stimulating the heart, for sensing cardiac activity, and/or for stimulating other tissues. The stimulation electrodes beneficially can be implanted using minimally or non-invasive techniques, thus avoiding the complex, higher-risk procedures associated with traditional implantation and stimulation techniques. In certain embodiments, the pulse generator also can be implanted within a patient's esophagus or, alternatively, a patient's airway, using minimally or non-invasive techniques.

In one aspect, an implantable cardiac stimulation system is provided that may include one or more electrodes carried by one or more electrical leads, implantable within a patient's upper gastrointestinal tract. The electrical leads may be attachable to an implantable pulse generator for generating and delivering electrical stimulation signal to the electrodes, and optionally for receiving electrical signals from one or more electrodes representing sensed parameters. The pulse generator may be housed in a control housing, which may be implanted within the patient's upper gastrointestinal tract, for example in the patient's esophagus. In some embodiments, the pulse generator may be implanted subcutaneously, for example in the patient's pectoral region or affixed to the external wall of the esophagus, or may be implanted within the patient's trachea, or within the patient's pharynx or nasal cavity.

The electrical stimulation signal generated by the implantable cardiac stimulation system may be effective for performing atrial cardiac pacing, ventricular cardiac pacing, dual chamber cardiac pacing, cardiac defibrillation, cardioversion, cardiac resynchronization therapy, gastric electrical stimulation, or a combination thereof. As used herein, the terms “electrical stimulation signal,” “electrical signal,” and “signal” are used interchangeably and may generally refer to any transmittable electrical current, and are not limited to a transmission containing information or data. As used herein, the terms “electrical pulse” or “pulse” are used interchangeably and may generally refer to one or more intermittent transmissions of an electrical current, such as is used during cardiac synchronization therapy. In addition, the implantable cardiac stimulation system may be operable to sense cardiac electrical activity, other cardiac activity, or other physiological parameters, and to generate and deliver electrical stimulation pulses in response thereto. Accordingly, the devices and methods described herein may be employed to treat various cardiac symptoms, such as asystole, bradycardia resulting from, for example, bilateral bundle branch block, bifascicular block, and first, second, and third degree atrioventricular block, tachyarrhythmia, tachycardia, and congestive heart failure. The devices and methods described herein may further be employed to support surgical anesthesia procedures and cardiac procedures. In certain embodiments, the devices may be operated in cooperation with additional conventionally implanted electrodes, for example, transvenous electrodes, epicardial electrodes, or epidermally or subcutaneously placeable electrodes.

In another aspect, implantable system and devices are provided for stimulation of essentially any tissue structure accessible via a patient's upper gastrointestinal tract or airway. That is, the upper gastrointestinal tract or airway may be used to position one or more electrodes for stimulating tissue structures other than the heart. For example, the phrenic nerve may be stimulated to activate the diaphragm or the diaphragm may be directly stimulated, to provide therapy to patient's suffering from respiratory paralysis. In another example, stomach nerves and smooth muscles may be stimulated for treating eating or digestive disorders and related conditions, such as obesity, gastroparesis, nausea, or vomiting, without requiring invasive electrode delivery required by conventional systems.

As used herein, the terms “comprise,” “comprising,” “include,” and “including” are intended to be open, non-limiting terms, unless the contrary is expressly indicated.

Like numbers refer to like elements throughout the following description.

I. Description of Anatomy

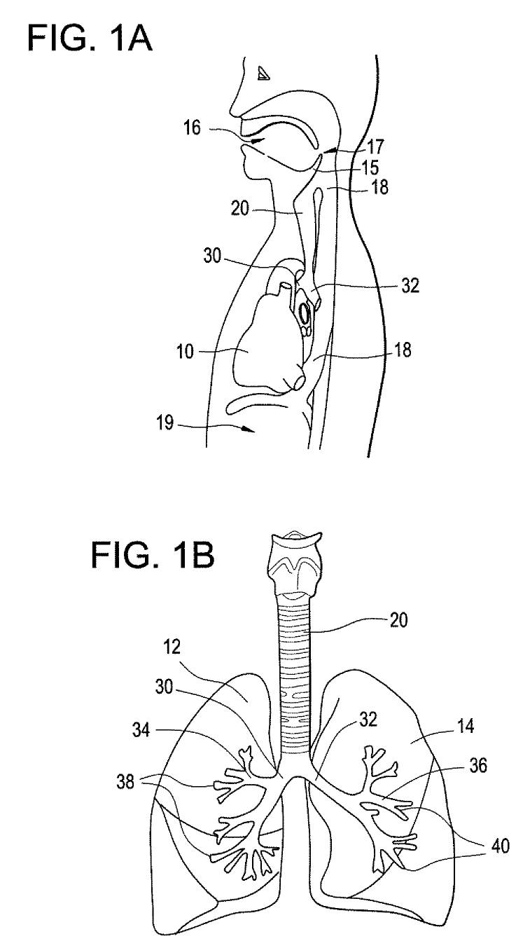

FIG. 1A illustrates a partial anatomical view of a human upper gastrointestinal tract, pulmonary system, and cardiovascular system, representing the relative positions of heart 10, the upper gastrointestinal tract, the trachea 20, and the bronchi 30, 32. The upper gastrointestinal tract includes the mouth 16, the pharynx 17, the esophagus 18, and the stomach 19. The heart 10 is positioned substantially anterior to the trachea 20 and bronchi 30, 32, which are substantially anterior to the esophagus 18. After the bronchial branch, the esophagus 18 is positioned substantially posterior to the heart 10. Accordingly, electrodes positioned at selected positions within the upper gastrointestinal tract or the pulmonary system can be in proximity to certain areas of the heart 10.

The epiglottis 15 directs flow between the digestive system and the pulmonary system. The human pulmonary system includes the trachea 20 and bronchial tree, which includes the bronchi and bronchioles. Each time one of these airways branches (e.g., splits into two or three), it forms a new generation of airway. FIG. 1B illustrates an anatomical view of a pulmonary system that includes the trachea 20 (0 generation airway), and the right lung 12 and the left lung 14. The trachea 20 branches into the right primary bronchus 30 and left primary bronchus 32 (first generation airways), which in turn branch into the lobar bronchi (second generation airways). The lobar bronchi include three right secondary bronchi 34 and two left secondary bronchi 36. The secondary bronchi 34, 36 branch into the bronchopulmonary (third generation airways), which includes, as shown in the Figure, the right tertiary bronchi 38 and left tertiary bronchi 40. Although not shown in FIG. 1B, the tertiary bronchi 38, 40 branch into primary bronchioles (fourth generation airways) and ultimately into terminal bronchioles which are associated with alveoli for facilitating gas exchange in the lungs. The diameter of the bronchi is typically approximately seven to eleven mm at the primary bronchi, and progressively decreases down the segmental bronchi to a diameter of less than about one millimeter at the bronchioles.

FIGS. 1A and 1B demonstrate that the upper gastrointestinal tract and the pulmonary system provide access in proximity to various areas of the heart 10. The terms “upper gastrointestinal tract,” “gastrointestinal tract,” and “digestive system” as used herein may be used interchangeably and refer to components of the upper gastrointestinal tract, including, but not limited to, the mouth 16, pharynx 17, esophagus 18, and stomach 19. The terms “airway” and “pulmonary system” as used herein may be used interchangeably and may refer to the bronchi and/or the trachea 20. The terms “bronchus,” “bronchi,” and “bronchial tree” as used herein may be used interchangeably and may refer to any of the individual components of the bronchi, including the primary bronchi 30, 32, the secondary bronchi 34, 36, the tertiary bronchi 38, 40, and/or the bronchioles branching therefrom.

Since the esophagus is positioned just posterior to the heart and the stomach just inferior, electrode positioning within the esophagus or the stomach operate in a manner similar to an epicardial electrode placed on the heart. Similarly, proximity of the bronchi to the heart, separated by the very thin tissues of the pericardium and lung pleura, provide for electrodes positioned therein to operate in a manner similar to an epicardial electrodes. In addition, the anatomy of the upper gastrointestinal tract and the airway provide a less invasive method for positioning electrodes within upper gastrointestinal tract or the airway to stimulate a patient's gastric system, such as the nerves and or muscles of the esophagus or the stomach. In one example, one or more electrodes positioned within a patient's upper airway, such as the nasal cavity, trachea, or primary bronchi, or within a patient's upper gastrointestinal tract, such as the pharynx or esophagus, may be used to stimulate nerves in proximity to the airway and/or gastrointestinal tract, such as the vagus nerve and its respective branches, or the baroreflex system and its barorecepters and/or nerves in the auricles of the heart, vena cavae, carotid sinus, and aortic arch. Accordingly, positioning an electrode and/or a pulse generator in a patient's upper gastrointestinal tract or airway in proximity to the heart provides a minimally or non-invasive technique for implanting cardiac or other tissue stimulation and/or sensing devices, and avoids the complexity and inherent risks of traditional techniques requiring complex, invasive procedures for implantation. Furthermore, because the peristaltic action of the esophagus and stomach and the slow breathing rate of the lungs as compared to the beating heart rate (e.g., approximately 12 breaths per minute compared to approximately 70 heart beats per minute), an electrical lead and electrode implanted within the upper gastrointestinal tract or airway advantageously would suffer much less mechanical fatigue and stress than a transvenous or epicardial lead, and thus should be less prone to mechanical failure.

II. Implantable Electrodes and Electrical Leads Attachable to a Pulse Generator Implantable in an Upper Gastrointestinal Tract or Airway

An implantable stimulation device, dimensioned to be implantable in a patient's upper gastrointestinal tract or airway, can include one or more electrodes also implantable at one or more positions within the gastrointestinal tract or the airway. The electrodes can be positioned and fixed at different areas within the upper gastrointestinal tract and, optionally, the airway, to electrically stimulate various cardiac components, including the sinoatrial node, the vagus nerve, the right atrium, the right ventricle, the left atrium, and the left ventricle. The electrical stimulation can be for administering cardiac therapy, such as for performing atrial cardiac pacing, ventricular cardiac pacing, dual chamber cardiac pacing, cardiac defibrillation, cardioversion, cardiac resynchronization therapy, or a combination thereof, depending upon the electrode configuration. Employing multiple electrodes in combination may optimize the path of electrical current, thus improving treatment and minimizing current or voltage requirements to achieve the intended therapy. In some embodiments, implantable stimulation device may further be operable to sense cardiac activity with one or more of the implantable electrodes to aid in administering cardiac therapy, as more fully described herein. In another example, the one or more electrodes positioned within the upper gastrointestinal tract may be used to directly stimulate other tissues or organs, such as the phrenic nerve, the diaphragm, or nerves and smooth muscles of the stomach.

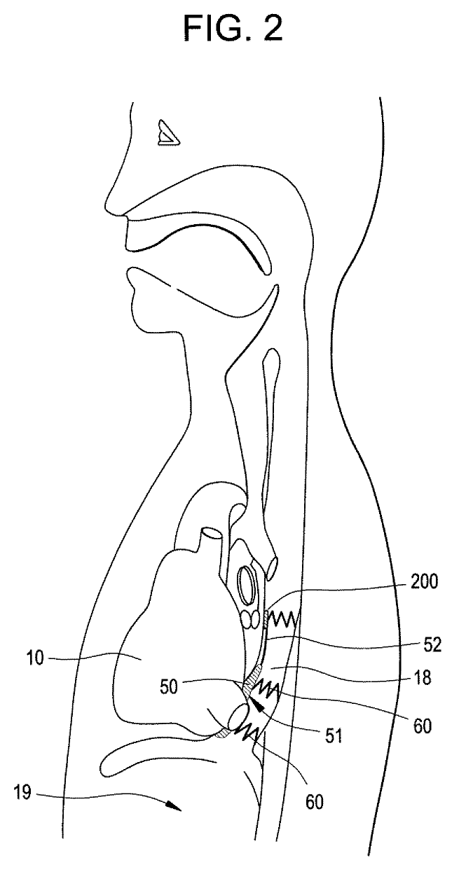

FIG. 2 illustrates one embodiment of an implantable stimulation system. A controller housing 50 including a pulse generator 51 may be adaptable for implantation at a selected housing position within the upper gastrointestinal tract, such as within the esophagus 18. In one embodiment, the controller housing 50 may be retained by one or more anchor devices 60, as is more fully described herein. In other embodiments, the controller housing 50 and pulse generator 51 may be implantable at other points in the upper gastrointestinal tract, such as within the stomach 19. A controller housing 50 implanted within the stomach 19 may be retained by one or more anchor devices that secure the controller housing 50 to the stomach 19 and prevent migration from the housing implantation position. Representative examples of such retention devices may include hooks, barbs, suture, staples, adhesive, foldable umbrella, inflatable balloon, fillable semi-permeable membrane, or any combination thereof.

As used herein the term “controller housing” generally refers to the structure or casing that houses the pulse generator and any other electronic circuitry, hardware, software, and for performing electrical stimulation and sensing as described herein. As used herein, the term “pulse generator” generally refers to any device operable of generating electrical stimulation signals, such as an electrical current; though, in some embodiments, a “pulse generator” may also be operable for receiving electrical signals representing sensed or measured parameters from one or more sensing electrodes or other sensors. Accordingly, a “pulse generator” as referred to herein may generate electrical pulses or shocks, receive electrical signals, or both. Furthermore, when referencing the “controller housing,” the “pulse generator” is included therein. In one embodiment, the controller housing 50 may have at least one electrode affixed to, or integrated with, its outer casing and in electrical communication with the pulse generator. The electrode controller housing electrode may be positioned at a point on the controller housing that will be positionable so as to contact the inner wall of the esophagus 18 upon implantation of the controller housing 50. Accordingly, the controller housing electrode may be operable to deliver electrical stimulation signals, such as electrical pacing or electrical shock signals to administer the desired therapy, as further described herein.

In another embodiment, as illustrated in FIG. 2, the controller 50 housing and the pulse generator 51 may be electrically coupled to at least one electrical lead 52. At least one electrode 200 is affixed to, integrated within, or carried by an electrical lead 52. The one or more electrodes 200 carried by one or more electrical leads 52 may be used in addition to, or instead of, an electrode affixed to or integrated with the controller housing 50. The electrode 200 may be positionable and fixable to a point within the upper gastrointestinal tract, for example to the epithelial tissue of the esophagus's 18 inner wall, such that the electrode 200 is in proximity to an area of the heart 10 to which electrical stimulation is to be delivered. The electrode 200 and/or the electrical lead 52 may be fixed to the inner wall of the gastrointestinal tract by one or more anchor devices, as further described herein. An implantable stimulation device according to this embodiment may be used to administer pacing therapy to the heart 10 using the electrode 200, such as left ventricular pacing. In one embodiment, one or more electrodes 200 may be positionable and fixable at a point near or within the patient's stomach 19, such as may be used to stimulate a patient's gastric system, for example, when treating eating or digestive disorders.

As used herein, the term “carried” when referring to an electrical lead carrying an electrode includes electrodes temporarily or permanently affixed to the electrical lead, electrodes integrated within the electrical lead such that they are a single component, or the like. The electrodes described by various embodiments herein may be positioned at or near the distal end of the electrical lead, as illustrated in FIG. 2. However, in other embodiments, an electrode may be positioned proximal to the distal end of the electrical lead, for example, in embodiments including a single electrical lead that carries more than one electrode, such as one at or near the distal portion of the lead and one or more electrodes on the same electrical lead proximal to the distal electrode. Yet in other embodiments, the pulse generator may communicate wirelessly with one or more electrodes or other sensors, and thus an electrical lead is not required.

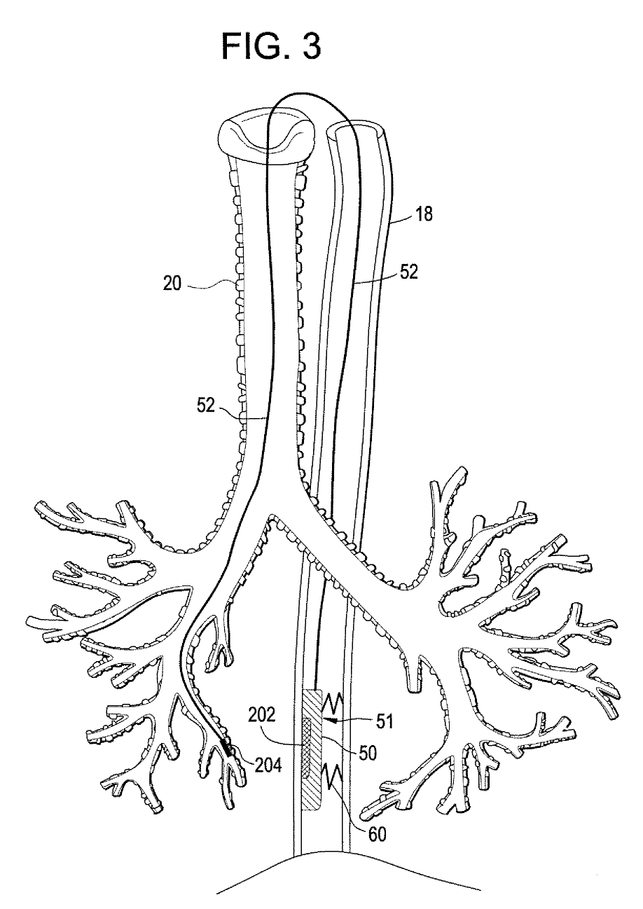

FIG. 3 illustrates another embodiment of an implantable stimulation system, having a controller housing 50 that includes a pulse generator 51, at least one electrode implantable within a patient's upper gastrointestinal tract, and at least one electrode implantable within the patient's airway. The controller housing 50 may be retained by one or more anchor devices 60, as is more fully described herein. In this embodiment, the controller housing 50 includes a controller electrode 202 affixed to or integrated with the casing of the controller housing 50. At least one electrical lead 52 is electrically coupled to the pulse generator 51 and passes from the controller housing 50 within the upper gastrointestinal tract, such as the esophagus 18′ and passable to a position within the patient's airway. The electrical lead 52 carries at least one electrode 204, which is positionable and fixable at a position within the patient's airway, such as within the right or left primary, secondary, or tertiary bronchi, the bronchioles, or a combination thereof. While FIG. 3 illustrates only a single electrical lead 52 and electrode 204 implantable within the airway, multiple electrical leads 52 each carrying at least one electrode 204 may be coupled to the pulse generator 51 and implantable at various positions within the airway.

This embodiment can be operable to administer defibrillation therapy by delivering a defibrillation signal between the controller electrode 202 implanted in the esophagus and the electrode 204 implanted within the airway. Additional cardiac therapies, such as pacing, cardioversion, dual chamber pacing, or cardiac resynchronization therapy, may be administered through this embodiment, delivering electrical stimulation signals and, optionally sensing, by way of the one or more electrodes 204 implanted in the airway and the controller electrode 202.

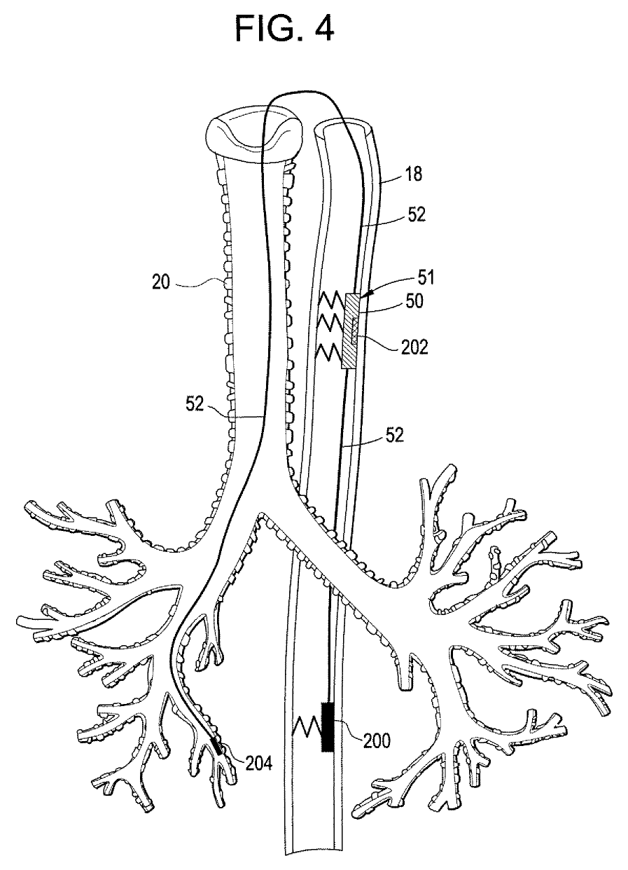

FIG. 4 illustrates another embodiment of an implantable stimulation system, which includes a controller housing 50 with a pulse generator 51 implantable within a patient's upper gastrointestinal tract, such as within the esophagus 18. The controller housing 50 may be retained by one or more anchor devices 60, as more fully described herein. One electrical lead 52 electrically coupled to the pulse generator 51 carries an electrode 200 that is positionable and fixable at a position within the esophagus 18, or other area within the gastrointestinal tract. A second electrical lead 52 carries an airway electrode 204 which is positionable and fixable at a position within the airway, such as in the bronchus, the bronchioles, or a branch thereof. Furthermore, the controller housing 50 of this embodiment may include a controller electrode 202, as described herein. An implantable system according to this embodiment is operable to deliver electrical stimulation signals to the esophageal electrode 200, the airway electrode 204, and the controller electrode 202, and is further operable to deliver one or more defibrillation signals between any two electrodes 200, 202, 204.

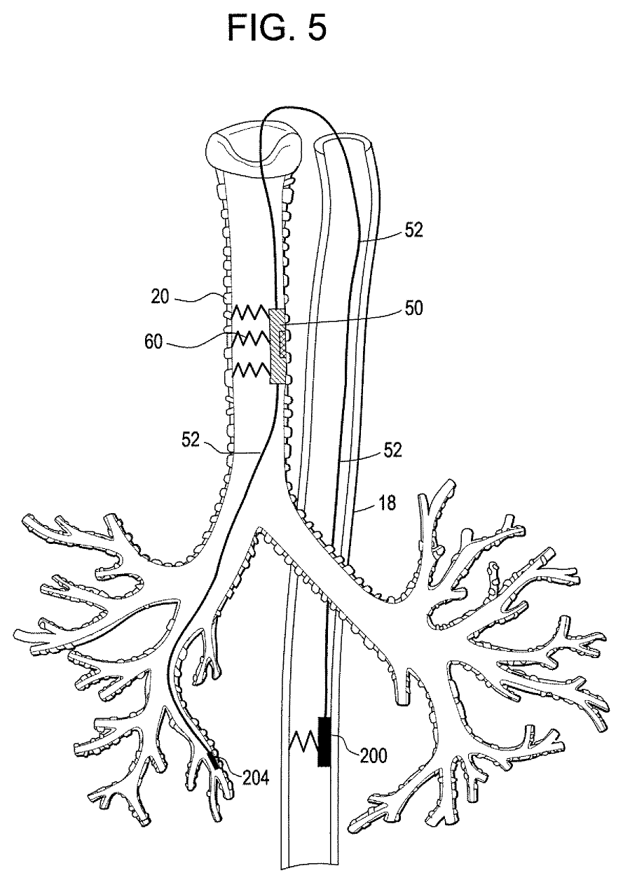

In another embodiment, illustrated in FIG. 5, the controller housing 50 may be implantable within a patient's airway, such as within the trachea 20. The controller housing 50 may be retained within the airway by one or more anchor devices 60, as more fully described herein. Similar to embodiments described with reference to FIG. 4, this embodiment may include at least one electrical lead 52 carrying at least one electrode 204 to a position within the airway, such as within the bronchi, bronchioles, or a branch thereof. This embodiment may also include a second electrical lead 52 carrying at least one electrode 200 to a position within the upper gastrointestinal tract, such as the esophagus 18. The controller housing 50 of this embodiment optionally may include a controller electrode for use in administering the desired therapy.

FIG. 6 illustrates another embodiment of an implantable stimulation system, which includes a controller housing 50 with a pulse generator 51 implantable within a patient's upper gastrointestinal tract, such as within the esophagus 18. The controller housing 50 may be retained by one or more anchor devices 60, as more fully described herein. In one embodiment, at least one electrical lead 52 electrically coupled to the pulse generator 51 may carry at least one electrode 240 that is positionable and fixable at a position at a position on the epicardium of a patient's heart 10 by passing through an aperture 242 formed in the upper gastrointestinal tract. The electrode 240 may be delivered by any delivery means described herein, such as using a delivery device like an endoscope or a catheter. The electrode 240 may be similar to conventional epicardial electrodes and may be affixed to the epicardium in any conventional manner. In another embodiment, the electrode 240 may be configured as a needle electrode, which may penetrate the wall of the esophagus 18 and directly couple to the epicardium of the heart 10. In embodiments including more than one electrode 240, one aperture 242 may be formed for each electrical lead 52 carrying the electrodes 240, as is illustrated in FIG. 6. However, in other embodiments, the electrical leads 52 may be bundled or integrated as a single lead from the controller housing and split after passing through a single aperture 242. A cannula, such as those described herein, may be implanted within the aperture or apertures 242 formed in the esophagus. In some embodiments, the controller housing 50 may include a controller electrode, as described herein.

The embodiments illustrated in FIGS. 2-6 are provided for exemplary purposes and other suitable electrode and controller housing configurations are envisioned. For example, other electrode placements within the esophagus, stomach, bronchi, or trachea, or any combinations of those described herein, may also be employed to perform cardiac or other tissue stimulation and/or sensing. Moreover, in other embodiments, electrodes positionable in a patient's gastrointestinal tract or airway may be used in combination with one or more conventionally implanted electrodes, such as transvenous electrodes, epicardial electrodes, or epidermally or subcutaneously placeable electrodes. In one embodiment, conventional transvenous leads may be used to perform sensing, right or left side pacing, and/or defibrillation, while esophagus or airway implanted electrodes may be use to perform ventricular pacing of the opposite side.

In embodiments including a single electrode, whether a controller electrode, an esophageal electrode, or an airway electrode, the implantable device may be operable for monitoring cardiac electrical activity and for identifying heart deficiencies, including, for example, bradycardia, ventricular tachycaria, or atrial fibrillation. A single electrode device also may be used for pacing the heart right atrium by delivering appropriately timed electrical pulses from the pulse generator in a manner similar to that as performed by conventional right atrial pacemakers. The single implanted electrode may additionally be used to treat atrial fibrillation by delivering a low voltage, high pacing rate electrical pulse from the pulse generator, similar to anti-tachycardia pacing (“ATP”), or by delivering a high voltage electrical excitation from the pulse generator.

In embodiments including at least two electrodes, for example one implanted in proximity to the right atrium and one in proximity to the right ventricle, the implantable device may be operable to perform cardiac pacing of the right atrium and the right ventricle with an appropriate delay (A-V delay) between them, which may be generally be referred to as dual-chamber pacing. Two electrode embodiments may be operable to perform any conventional dual-chamber pacing, such as DDDR pacing, for example. Cardiac defibrillation may also be performed by implantable devices containing at least two electrodes, with one delivering the signal and the other acting as the counter electrode, creating a shock vector therebetween. Two electrode embodiments may further be operable to provide atrial fibrillation therapy, such as by anti-tachycardia pacing therapy to the left atrium and an optional defibrillation pulse signal to treat (i.e., stop) atrial fibrillation. In one embodiment, two electrodes may be carried by a single electrical lead, or one electrode may be carried by an electrical lead and the other may be a controller electrode.

An implantable device including at least three electrodes, for example a controller electrode, an esophageal electrode, and an airway electrode, such as is illustrated in FIG. 4, can be operable to perform cardiac resynchronization therapy, for example having electrodes in proximity to the left ventricle, right ventricle, and the right atrium. A three electrode embodiment may also be operable to perform cardiac resynchronization therapy defibrillation (“CRT-D”), in which defibrillation pulsing is added to the dual-chamber pacing functions, and two shock vectors can be created. Furthermore, a three-electrode embodiment may simply perform cardioversion/defibrillation therapy (such as that performed by conventional implantable cardioverter-defibrillators), and may optionally perform cardiac pacing therapy. Two distally positioned electrodes may perform the defibrillation functions and a proximally placed electrode near the right atrium may only be necessary if additional cardiac pacing therapy is provided. A third electrode, however, may alternatively be operable to perform sensing functions, such as sensing cardiac electrical activity, and/or to reduce the electrical energy required for defibrillation by supplementing the stimulation signal delivered.

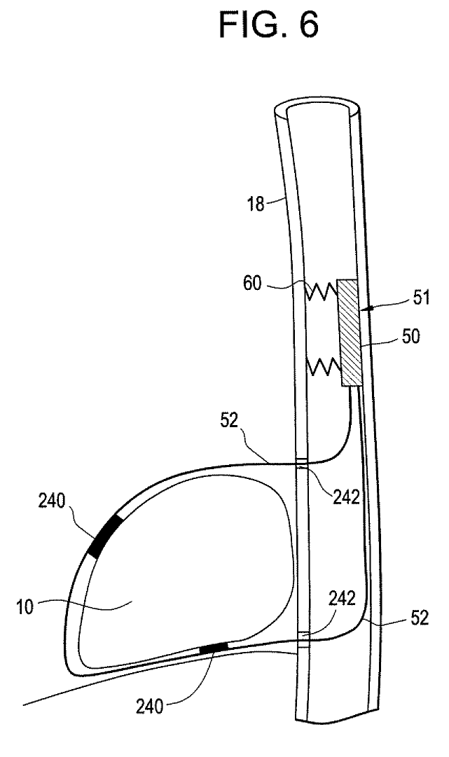

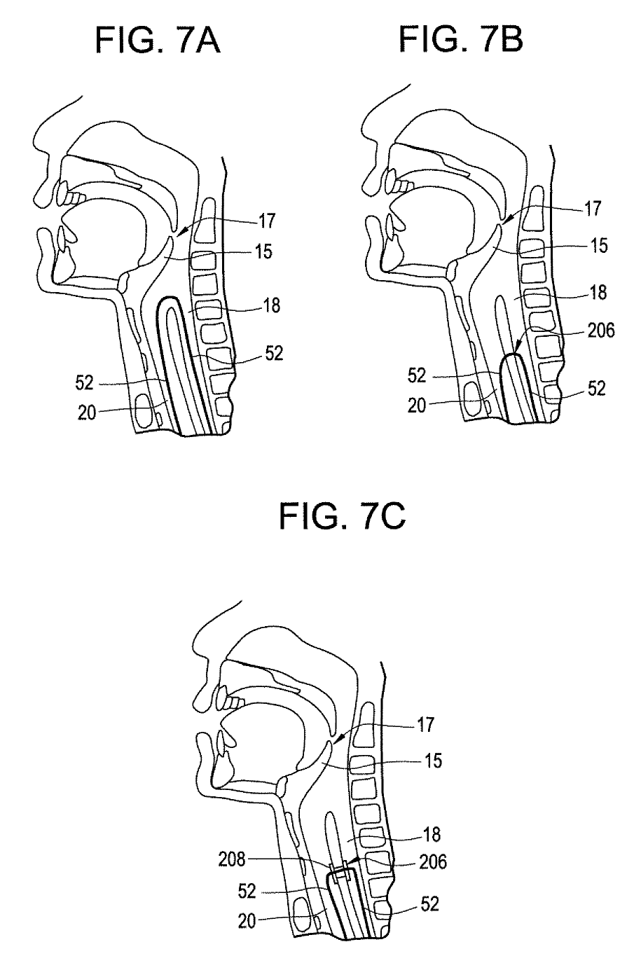

FIGS. 7A-7C illustrate embodiments for passing the one or more electrical leads 52 from within the upper gastrointestinal tract to the airway. The embodiment shown in FIG. 7A includes an electrical lead 52 passing from the esophagus 18, through the pharynx 17, under the epiglottis 15, and into the trachea 20. An electrical lead 52 according to this embodiment may be dimensioned so as to minimally interfere with the functioning of the epiglottis 15. For example, the electrical lead 52 may have a substantially flat cross-section at least at the point passing under the epiglottis. In another example, the electrical lead 52 may be pre-formed to have a substantially “U” shape, such that it approximately follows the patient's anatomy at the junction of the esophagus and the trachea near the epiglottis. A pre-formed electrical lead may have a portion that is more rigid than others, such that it may be formed and substantially maintain its shape. In one example, the pre-formed portion may be at least partially pliable, such that it may be formable by an operator, allowing for adjustments based on the anatomy of the patient receiving the implant. In another example, the electrical lead 52 may be positionable such that it may be free to move upon the movement of the epiglottis, for example by at least partially anchoring the electrical lead at or near the proximal end of the esophagus and at or near the proximal end of the trachea, maintaining sufficient length between the two anchor points to provide moveable slack. The embodiment illustrated in FIG. 7A provides a noninvasive way to pass the electrical lead 52 between the gastrointestinal tract and the airway.

In another embodiment, illustrated in FIG. 7B, the one or more electrical leads 52 may be passed through one or more apertures 206 formed through the walls of the esophagus 18 and the trachea 20. In embodiments including multiple electrical leads 52, they may either be bundled together and passed through a single aperture 206, or fewer than all may pass through multiple apertures 206, such as forming one aperture 206 for each electrical lead 52.

In another embodiment, illustrated in FIG. 7C, similar to that described with reference to FIG. 7B, one or more apertures 206 are formed through the esophagus 18 and the trachea 18 for passing one or more electrical leads 52 therethrough. However, one or more cannulae 208 may additionally be implanted at or within the aperture or apertures 206, as further described herein with reference to FIGS. 14-16. The cannula 208 may aid in sealing the thoracic cavity, the esophagus, and the airway from each other, to exclude the passage of air, biological contaminants, or biological fluids therebetween, to provide structural integrity to the aperture or apertures 206 the tissue walls, to house the electrical leads 52 to ease movement therethrough, and to reduce irritation, inflammation, or infection where the electrical leads 52 may otherwise contact the walls of the upper gastrointestinal tract or the airway.

Controller Housing

The controller housing 50 may be implantable within a patient's upper gastrointestinal tract, such as within the esophagus 18, as illustrated in FIG. 2, or optionally within the trachea 20, as illustrated in FIG. 5. In one embodiment, the controller housing 50 may be dimensioned to be implanted further down the airway, such as the secondary or tertiary bronchi. As described above, the pulse generator 51 may be operable to generate and deliver electrical stimulation signals to the one or more electrodes 200, 202, 204 via the electrical leads 52, or wirelessly, effective in performing cardiac pacing, cardiac defibrillation, cardioversion, cardiac resynchronization therapy, or a combination thereof. In one embodiment, the pulse generator 51 may be operable to measure physiological conditions via one or more of the electrodes 200, 202, 204, such as measuring electrical cardiac activity like electrical impedance across two points, or other cardiac activity. In another embodiment, the pulse generator 51 may be operable to control and receive measurements representing other physiological parameters from additional sensors implanted within the patient's body, such as an accelerometer, a strain gauge, a pressure transducer, or a temperature sensor. Although various embodiments described herein include a single controller housing and pulse generator performing all of the cardiac stimulation and sensing functions, other embodiments may include more than one controller housing and pulse generator, with each pulse generator performing separate functions and/or providing redundant functioning for reliability and safety purposes.

A controller housing 50 implantable in an upper gastrointestinal tract, or optionally an airway, is proportioned to substantially permit solid, fluid, or gas flow through the respective passageway past the controller housing and to avoid substantial discomfort to the patient. For example, for an embodiment in which the controller housing 50 is implantable in the esophagus 18, an esophagus may distend to an inner diameter up to approximately 3 cm and have a length of approximately 18 to 26 cm; thus, the controller housing 50 may be proportioned to be smaller in diameter than approximately 3 cm and have a length less than approximately 18 to 26 cm. For example, in one embodiment the controller housing 50 is an elongated cylinder with a diameter of approximately 4 to 10 mm, and a length of approximately 4 to 11 cm. In another embodiment, the controller housing 50 has a diameter less than approximately 7 mm and a length of less than approximately 6 cm.

In another embodiment in which the controller housing 50 is implantable in the trachea 20, a trachea may have an inner diameter of approximately 15 to 25 mm and a length of approximately 10 to 16 cm; thus, the controller housing 50 may be proportioned to be smaller in diameter than 15 to 25 mm and have a length less than approximately 10 to 16 cm. For example, in one embodiment the controller housing 50 is an elongated cylinder with a diameter of approximately 4 to 10 mm, and a length of approximately 4 to 11 cm. In another embodiment, the controller housing 50 has a diameter less than approximately 7 mm and a length of less than approximately 6 cm.

In certain embodiments, the cross section of the controller housing 50 may be substantially curvilinear, such as circular or elliptical; though in other embodiments, the cross section may be substantially square, rectangular, triangular, or the like. The controller housing 50 may further be proportioned such that at least part of the controller housing 50 will substantially contact the inner wall of the gastrointestinal tract or airway at the selected implantation site. Thus, in embodiments in which the controller housing 50 has a curvilinear cross section, the radius of curvature may approximate that of the inner wall of the upper gastrointestinal tract or the airway.

In addition, the controller housing embodiments described herein may further optionally include radiopaque material to aid in delivery procedures using imaging techniques, biocompatible coating, medicinal or therapeutic coating, such as anti-proliferative agents, steroids, antibiotic agents, or any combination thereof.

Anchor Devices

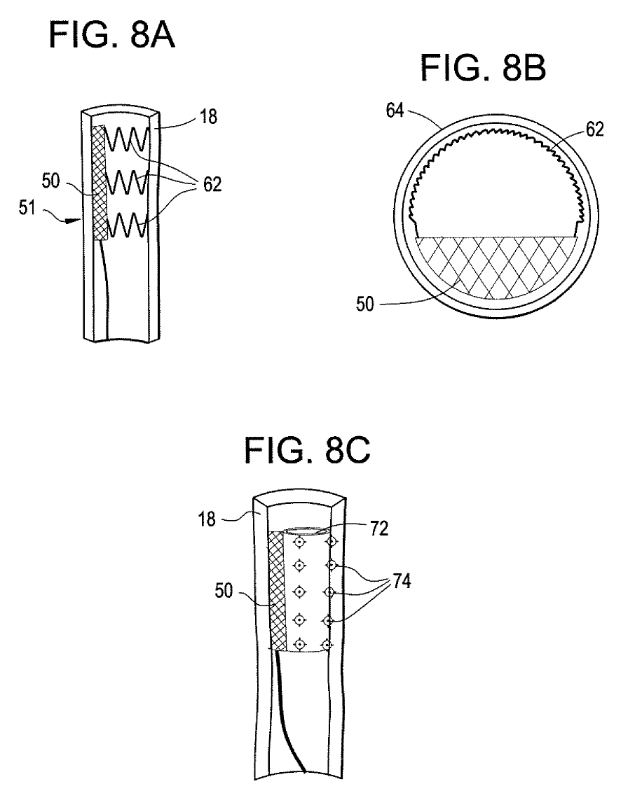

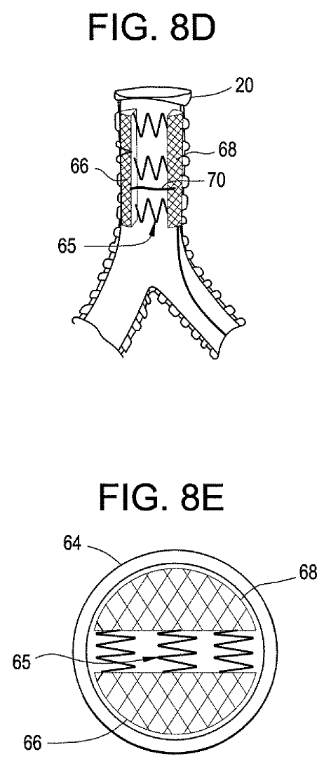

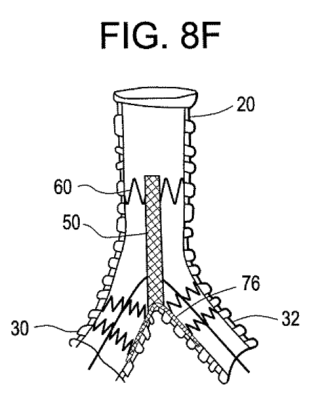

FIGS. 8A-8E illustrate several devices for anchoring the controller housing containing the pulse generator within the upper gastrointestinal tract or the airway, such as the esophagus or the trachea, according to certain exemplary embodiments. These embodiments are representative; other means for positioning and anchoring the controller housing are envisioned and may be employed. Although at least some of the embodiments of the anchor device may be described as being implantable within the esophagus, the anchor device designs equally apply to controller housing implantable at other positions of the upper gastrointestinal tract, or within the trachea or bronchus.

FIG. 8A illustrates an extensible member, such as a radially expandable member 62 for anchoring the controller housing containing the pulse generator 51 to the inner wall of the esophagus 18, for example to the epithelial tissue. The expandable member 62 may be configured in a tubular shape similar to a stent, such that it includes at least one circumferential ring extending from the controller housing 50 and proportioned to interface with the inner wall of the esophagus 18. For example, a stent-like expandable member 62 may have a interwoven, zigzag, wave-like, mesh, z-shaped, helical, or otherwise radially expandable and contractible or collapsible shape as is known, and may extend from one side of the controller housing 50. In its expanded or extended position, the expandable member 62 may be have a radius of curvature substantially similar to the inner wall of the esophagus 18 so as to promote retention of the controller housing 50. Furthermore, an expandable member 62 configured similar to a stent, having a hollow lumen or path existing along its axis, is advantageously proportioned to permit gas, solid, and fluid flow through the esophagus 18 and around the controller housing 50 at the selected housing implantation site or position. The expandable member 62 may be formed from metals, such as nickel-titanium alloys, stainless steels, tantalum, titanium, gold, cobalt chromium alloys, cobalt chromium nickel alloys (e.g., Nitinol™, Elgiloy™) or from polymers, such as silicone, polyurethane, polyester, or any combination thereof. Available esophageal stents, such as the Ultraflex™ Esophageal Stent or the Polyflex® Esophageal Stent (Boston Scientific Corporation (Natick, Mass.)). In other embodiments, the anchor device may be formed as a substantially solid tubular member, and may include a separate expansion means for extending from the controller housing 50 and exerting force against the tubular member, causing the tubular member to substantially engage the inner wall of the trachea 20. Each embodiment of the expandable member 62 as described provides sufficient force against the esophagus 18 for retaining the controller housing 50 in place, although it should not apply excessive force, so as to avoid damaging the esophagus 18 or causing discomfort to the patient. Expansion of these expandable member 62 embodiments may be performed mechanically, such as by stent-like designs, springs, inflatable cuffs, or the like, or may be caused by the characteristics of the members, such as shape memory alloys, or any combination thereof. Optionally, any of the described expandable members 62 may further include one or more barbs, hooks, suture, and the like for securing the member 62 to the inner wall of the esophagus 18.

During implantation of the controller housing 50, the stent-like expandable member 62 may be contracted or collapsed against or within close proximity to the controller housing 50 to minimize the profile during delivery, for example when delivered using a delivery device 64, such as a catheter or other elongated lumen for delivery, as is illustrated in FIG. 8B. Upon positioning the controller housing 50 at the implantation site and releasing it from the delivery device 64, the expandable member 62 expands and substantially engages the inner wall of the esophagus. The controller housing 50 may optionally include a recess or have a substantially flat surface on the side from which the expandable member 62 extends, such that the recess or flat surface receives at least part of the expandable member 62 when in compressed form, further reducing the profile to aid in delivery by a delivery device.

FIG. 8C illustrates another embodiment of an extensible member attached to the controller housing 50, configured as a tubular expandable member 72. The tubular expandable member 72 is substantially tubular in shape, similar to that as described with reference to FIGS. 8A and 8B, and may have solid or substantially solid tubular walls. The tubular expandable member 72 illustrated in FIG. 8C further includes one or more studs, barbs, hooks 74, or any combination thereof, to assist retaining the member 72 against the inner wall of the esophagus 18 at the selected housing implantation position. In certain embodiments, the tubular expandable member 72 may be formed from a polymer, such as silicone or polyurethane.

FIG. 8D illustrates another embodiment of an extensible member including one or more expandable connectors 65 connecting at least two separated controller housing sub-components 66, 68. Although better suited for implanting the controller housing within the trachea, because certain designs of the expandable connectors may obstruct solid flow through the esophagus, this embodiment may nonetheless be adaptable for anchoring a controller housing within the esophagus. In this embodiment, the one or more expandable connectors 65 create opposing forces against the two separated controller housing sub-components 66, 68, biasing each against the inner wall of the trachea 20 or esophagus at opposing areas, and thus anchoring the controller housing sub-components 66, 68 in place. The expandable connectors 65 may be configured similar to the radially expandable member 62 described with reference to FIGS. 8A-8B. In some embodiments, the expandable connectors 65 may be formed as a metallic or polymeric spring, from a shape memory alloy, as mechanically adjustable rigid members, or as telescoping members. In one embodiment, the two separate sub-components 66, 68 may be electrically coupled by one or more isolated electrical leads 70 to facilitate power transfer and/or electrical communication between the pulse generator circuitry existing within the controller housing sub-components 66, 68 and/or with one or more electrical leads carrying electrodes. The isolated electrical lead 70 may be integrated with the expandable connectors 65, such that it does not further obstruct the flow through the trachea or esophagus. Although the embodiment illustrated by FIG. 8C includes only two separated controller housing sub-components 66, 68, the controller housing may be formed as any number of controller housing sub-components, each being radially biased toward the inner wall of the trachea 20 or the esophagus.

Similar to the embodiment illustrated in FIGS. 8A and 8B, the two or more separated controller housing sub-components 66, 68 connected by one or more expandable connectors 65 may be compressed to a reduced profile during placement, for example, when delivering using a delivery device 64, as illustrated in FIG. 8E. In this embodiment, two separated controller housing sub-components 66, 68 each have a substantially semi-circular cross section and are complementary in shape to each other.

FIG. 8F illustrates an embodiment of an anchor device which may be employed to prevent distal migration of a controller housing 50 implanted within the trachea 20. According to this embodiment, the bifurcation between the trachea 20 and the right and left primary bronchus 30, 32 support the controller housing 50. An arched member 76 is attached to the distal end of the controller housing 50, includes an arm extending into each primary bronchus 30, 32, and is substantially supported by the bifurcation. The arched member 76 may be secured using an extensible member, such as any described with reference to FIGS. 5A-8E, or by any other anchoring means, such as a balloon, suture, staples, barbs, hooks, studs, adhesive, shape memory alloy members, or any combination thereof. In one embodiment, the arched member 76 may be shaped before or during implantation to more specifically follow the curvature of the bifurcation, farther facilitating retention of the controller housing 50 within the airway. In this embodiment, an additional anchor device 60 is affixed to the controller housing 50, such as those described herein. In other embodiments, anchor members may not be employed on either the controller housing, the arched member, or both.

Other embodiments of the anchor device for retaining the controller housing 50 at a desired position within the upper gastrointestinal tract or airway, though not illustrated, may be employed. For example, the controller housing may be held against the inner wall by suture, staples, adhesive, or a combination thereof, as is mown. In another embodiment, the anchor device may be a reversibly inflatable balloon, formed as a sleeve, having an opening passing axially therethrough, and expanding radially. In this example, the balloon may be deflated during placement and then inflated to expand radially by methods known, causing a biasing force against the esophagus or trachea. Further, the external surface of the balloon sleeve may be include texturing, texturing, suture, barbs, hooks, studs, adhesive, or any combination thereof, to further facilitate retaining the sleeve against the tissue wall. In yet another embodiment, the anchor device may be formed as one or more radially extending rigid members, which may be extensible, collapsible, telescoping, inflatable, formed from shape memory alloy, or the like, causing a radially biasing force against the inner wall of the esophagus or trachea.

In addition, any of the anchor devices described herein optionally may include a radiopaque material to aid in delivery procedures. The radiopaque material may be used in part or all of device. The radiopaque material may be useful to facilitate device or component placement using known imaging techniques. The anchor devices described herein optionally may include a biocompatible coating. The coating may include one or more prophylactic or therapeutic agents, such as anti-proliferative agents, steroids, antibiotic agents, or any combination thereof.

Pulse Generator

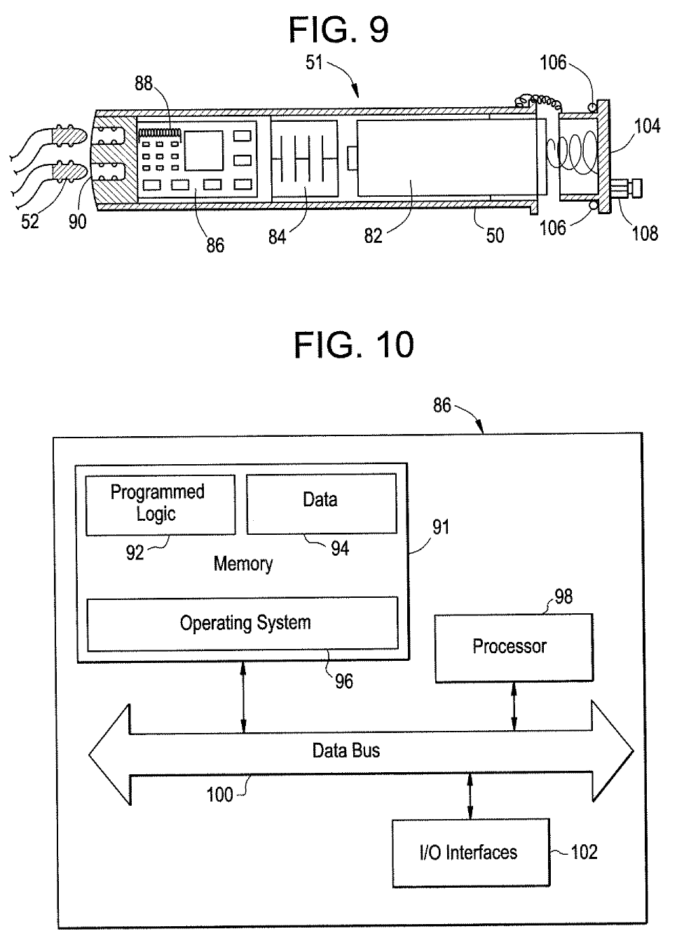

FIG. 9 illustrates an embodiment of a pulse generator 51 and many of the features that may be included as components of the pulse generator 51. Example features which may be included at least partially in a typical pulse generator 51 include a power source 82, a capacitor circuit 84, which may be used to charge and discharge during defibrillation, an electronic controller 86, which may be implemented by a microprocessor, an integrated circuit, a field programmable gate array (“FPGA”), or other electronic circuitry as is known, a communication module 88, one or more electrical lead sockets 90, and a controller housing 50 encapsulating components contained within the pulse generator 51 and forming the external structure thereof. As described herein, the pulse generator 51 and its associated elements, such as electrical circuitry, which may be at least partially provided within the controller housing 50, may be operable to generate and deliver an electrical stimulation signal, which may be effective for performing cardiac pacing, cardiac defibrillation, cardioversion, cardiac resynchronization therapy, or a combination thereof. The pulse generator 51 may optionally include electronic circuitry, hardware, and software to measure and sense electrical cardiac activity, other cardiac activity, other physiological parameters, or any combination thereof. Accordingly, the power source 82 in combination with the electronic controller 86 may include hardware and/or software suitable for delivering electrical stimulation signals, and optionally for receiving electrical sensing signals, via one or more electrical leads 52 electrically coupled to the lead sockets 90 and carrying one or more electrodes, as is described more fully herein.

Because the pulse generator 51 is implantable within an upper gastrointestinal tract or airway, such as an esophagus, trachea, or bronchus, or in some embodiments subcutaneously, the controller housing 50 may be constructed so as to withstand humidity, gasses, and biological fluids. The controller housing 50 may be hermetically sealed and constructed to withstand the environment and protect the circuitry, power source, and other elements contained therein. Controller housing 50 may be implantable in the esophagus, trachea, or bronchus, and at these locations it generally will not be continually immersed in a liquid environment, which in contrast to a subcutaneous implant. This characteristic enables the use of polymeric materials of construction. (In contrast, a subcutaneous implant often requires metallic materials of construction and laser or electron beam welded seams.) Accordingly, in embodiments implanted in the upper gastrointestinal tract or airway, the controller housing 50 may be entirely or partially constructed from one or more polymeric materials. Representative examples of suitable polymers include epoxy, polypropylene, polyethylene, polyamide, polyamide, polyxylene, polyvinyl chloride (“PVC”), polyurethane, polyetheretherketone (“PEEK”), polyethylene terephthalate (“PET”), liquid crystal polymer (“LCP”), and the like. In other examples, however, the controller housing 50 may be constructed from entirely or partially metallic materials. Representative examples include nickel, titanium, stainless steel, tantalum, titanium, gold, cobalt chromium alloy, or any combination thereof. In yet other examples, the controller housing 50 may be constructed from a combination of one or more of these polymeric or metallic materials. Other materials known in the art to be suitable for fabricating or encasing implantable medical devices also may be used to construct the controller housing 50.

All or partial polymeric construction of the controller housing 50 may be advantageous as compared to completely metallic construction, avoiding the Faraday cage effect that may be caused by a complete metallic casing. A Faraday cage effect may limit the use of electromagnetic fields to communicate or otherwise interface with the pulse generator 51. Accordingly, a non-conductive controller housing 50, such as one constructed from polymeric materials as described herein, allows electromagnetic fields for communicating with, controlling, and otherwise interfacing with the pulse generator 51. For example, electromagnetic fields may be used for recharging battery power sources associated with the pulse generator 51, without removing the pulse generator 51 and/or the battery power source. In another embodiment, the controller housing 50 may be constructed partially from metallic materials, for example at the points interfacing with the upper gastrointestinal tract or airway, and partially from polymeric materials. A controller housing 50 constructed in such a manner also avoids the Faraday cage effect by not being completely surrounded by an electrical conducting metal.

Some or all of the external components of the pulse generator 51, including the controller housing 50 and the anchor device as described with reference to FIGS. 5A-8F, may be entirely or partially coated to aid or improve hermeticity, electrical conductivity, electrical isolation, thermal insulation, biocompatibility, healing, or any combination thereof as is desired. Representative coatings include metals, polymers (e.g., polyxylene polymers, such as Parylene), ultra-nanocrystalline diamond, ceramic films (e.g., alumina or zirconia), medicinal agents, anti-inflammatory or anti-bacterial agents or coatings (e.g., silver coating), or combinations thereof. For example, the controller housing 50 may be coated by a polyxylene polymer to further electrically isolate the electrical circuitry, power source, and other elements from the patient's body. In another example, the controller housing 50 and/or the anchor device, particularly at the points interfacing with the gastrointestinal tract or airway, may be at least partially coated with a biocompatible coating, medicinal or therapeutic coating, such as anti-proliferative agents, steroids, antibiotic agents, or any combination thereof to promote healing of trauma caused during implantation and/or to avoid infection.

FIG. 10 depicts a schematic diagram of one example of an electronic controller 86 that may be utilized according to various embodiments in order to generate, deliver, receive, and/or process electrical stimulation and sensing signals to perform cardiac treatment. As used herein, the terms “electronic controller,” “electronic circuitry,” and “electrical circuitry” may be utilized interchangeably. The electronic controller 86 may be at least partially provided within the controller housing, such as illustrated in FIG. 9. The electronic controller 86 may include a memory 90 that stores programmed logic 92 (for example, software). The memory 91 may also include data 94 utilized in the operation of operating system 96 in some embodiments. For example, a processor 98 may utilize the operating system 96 to execute the programmed logic 92, and in doing so, may also utilize the data 94, which may either be stored data or data obtained through measurements or external inputs. A data bus 100 may provide communication between the memory 91 and the processor 98. Users may interface with the electronic controller 86 via one or more user interface device(s) 102, such as a keyboard, mouse, control panel, or any other devices suitable for communicating digital data to the electronic controller 86. The user interface device(s) 102 may communicate through wired communication, which may be removably coupled to the pulse generator during implantation or during servicing, or may communicate wirelessly, such as through radio frequency, magnetic, or acoustic telemetry, for example. The electronic controller 86 and the programmed logic implemented thereby may comprise software, hardware, firmware, or any combination thereof.

The elements of the pulse generator, for example the electronic controller 86, may be discrete components, or some or all elements may be based on VLSI technology, having many components embedded within a single semiconductor. In one embodiment, the electronic controller 86 is integrated with a flexible printed circuit board constructed from, for example, a polyimide film, e.g., Kapton™, (E.I. du Pont de Nemours & Co. (Wilmington, Del.)). A suitable electronic controller 86 may include more or less than all of the elements described herein. Although the electronic controller 86 illustrated in FIG. 10 is described as including each individual component internally within a single controller, multiple electronic controllers 86 may be employed, for example, each performing individual functions and/or each performing redundant functions of the other. Some of the components illustrated in FIG. 10 may exist external to the controller housing 50 and the patient, for example, within a separate processing unit, such as a personal computer or the like, in communication with the controller housing 50.

The power source 82 illustrated in FIG. 9 may be a battery of any known chemistry, for example a battery having high voltage, high capacity, low self-discharge, long durability, and that is non-toxic. Example battery chemistries may include lithium iodine, lithium thionyl chloride, lithium carbon monoflouride, lithium manganese oxide, and lithium/silver-vanadium-oxide. In another embodiment, the power source 82 may include one or more rechargeable batteries. Example rechargeable battery chemistries may include, but are not limited to, lithium ion, LiPON, nickel-cadmium, and nickel-metal hydride. The power source 82 may comprise more than one battery, for example a primary battery and a back-up battery, or in another example, certain pulse generator 51 elements may be powered by a first battery and certain other elements may be powered by a second battery.

Because the pulse generator 51 is implantable within the upper gastrointestinal tract or the airway, and thus relatively close to the patient's surface, a rechargeable power source 82 may be charged using electromagnetic charging, as known in the art. Other wireless charging methods may be used, for example, magnetic induction, radio frequency charging, or light energy charging. Embodiments including a rechargeable power source 82 may further be charged by direct charging, such as may be delivered by a catheter, through an endoscope, or an endotracheal tube, for example, to a charging receptacle 108, feedthrough, or other interface optionally included in the controller housing 50 and in electrical communication with the power source 82. The charging frequency and the charging duration of the power source 82 depends on its capacity and the device usage.

In another embodiment, the power source 82 may be replaceable, and the controller housing 50 may be adapted for simple, safe access to the power source, memory, processor, electrical circuitry, or other pulse generator elements, while implanted within the upper gastrointestinal tract or airway. For example, the embodiment illustrated in FIG. 9 optionally includes a reattachable detachable portion of the controller housing 50. In one embodiment, the detachable portion may be a replaceable removable cap 104 adaptable for removably connecting to the controller housing 50 of the controller housing 50. The removable cap 104 may create a hermetic seal between the main body of the controller housing 50 and the cap 104 using a flexible o-ring 106, for example. In some embodiments, the o-ring may be constructed from elastomeric polymers, such as perfluoroelastomer, silicone, acrylonitrile butadiene copolymers, butadiene rubber, butyl rubber, chlorosulfonated polyethylene, epichlorohydrin, ethylene propylene diene monomer, ethylene propylene monomer, or fluoroelastomers, or from soft metals, such as copper, gold, silver, tin, or indium. The removable cap 104 may be secured to the main body of the controller housing 50 by any fastener suitable for releasably securing two items, such as threads and threaded receiver, bolt, clamp, pin and slot, or adhesive, for example. In one embodiment, the removable cap 104 may be removably secured to the proximal end of the controller housing 50, providing easier access to the components contained therein. In another embodiment, the controller housing 50 may be adapted to include one or more removable caps 104 at other portions of the controller housing 50. The controller housing 50, may further include one or more recesses or protruding members to facilitate gripping the controller housing 50 during access and removal of the cap 104.

The removable cap 104 may also include means for forming an electrical contact with the power source 82, such as a standard spring, flat spring, or conical spring, such that when the cap 104 is removed the electrical contact is broken and no power is delivered to the pulse generator 51 from the power source 82. Accordingly, a controller housing 50 adapted to include a replaceable power source 82 allows for removing the cap 104, removing the power source 82, replacing the power source 82, re-securing the cap in a non-invasive, incisionless procedure, such as with the use of an endotracheal tube, a catheter, or an endoscope, for example. In one embodiment, the controller housing 50 may have a substantially elongated cylindrical shape and is dimensioned to allow commercially available batteries such as one or more “AAA-size,” “AAAA-size,” or button cell batteries having any of the battery chemistries described herein.

Other pulse generator 51 elements housed within the controller housing 50 may be accessible by a removable cap 104, and may be accessed and/or removed while the controller housing 50 remains implanted within the patient. For example, elements that may be accessed, maintained, or adjusted via a removable cap 104 may include sensors, communication antenna, hardware, software upgrades, lead sockets, circuitry, or memory.

In another embodiment, the reattachably detachable portion may be a sub-casing of the controller housing 50 that similarly provides access to one or more elements within the pulse generator. The sub-casing may be reattachably secured to the controller housing in a manner similar to that described with reference to the removable cap 104. For example, the sub-casing may provide an additional, sealed compartment, which may be in electrical communication with the remainder of the pulse generator 51. For example, the sub-casing creates a hermetic seal between the main body of the controller housing 50 and the sub-casing. The sub-casing may be secured to the main body of the controller housing 50 by any fastener suitable for releasably securing two items, such as threads and threaded receiver, bolt, clamp, pin and slot, or adhesive, for example. In one embodiment, the sub-casing may be removably secured to the proximal end of the controller housing 50, providing easier access to the components contained therein. With reference to FIG. 9, the removable cap 104 may be replaced by the detachable portion, such that instead of a cap, the detachable portion provides an additional, sealed compartment, which may be in electrical communication with the remainder of the pulse generator 51.

The one or more electrical lead sockets 90 illustrated in FIG. 9 may be an insulated, or otherwise electrically isolated, junction or feedthrough, enabling electrical communication between the one or more leads 52 and elements of the pulse generator 51, such as the electronic controller 86. As compared to conventional pulse generators implanted subcutaneously, typically requiring strict hermetic sealing, an upper gastrointestinal tract or airway implanted controller housings may be less demanding. Therefore, the lead socket or sockets 90 may simply consist of polymeric or elastomeric seals. However, more robust sealing mechanisms, such as a glass to metal sealed or a ceramic sealed feedthrough, may be used, such as for embodiments implantable subcutaneously. Any conventional fasteners may be used to secure (e.g., removably secure) an electrical lead 52 to a lead socket 90. Removably securing the electrical leads 52 to the controller housing 50 allows flexible implantation techniques. In other pulse generator 51 embodiments, however, the one or more electrical leads 52 may be permanently integrated with the controller housing 50, and thus may not be removable.

The pulse generator 51 and controller housing 50 may further include one or more sensors for monitoring conditions external to the patient's body. Being implantable within the upper gastrointestinal tract or the airway, the pulse generator 51 is substantially exposed to the environment external to the patient's body and may sense, measure, or record parameters substantially representative thereof. Example sensors include a pressure sensor for monitoring the air pressure within the esophagus or trachea and for evaluating the barometric pressure, or a temperature sensor for estimating temperature external to the patient's body. The measured air pressure in the esophagus or trachea may also be used for observing and/or recording parameters related to the patient's breathing, including, for example, respiration rate and airway pressure in the inspirium and expirium stages. Measurements related to breathing may help a physician detect, diagnose, and treat various chronic lung problems, such as asthma, bronchitis, emphysema, or chronic obstructive pulmonary disease, for example. These sensor devices and measured parameters are exemplary; other sensor devices may be operably associated with and/or mechanically connected to the pulse generator 51 and controller housing 50′ for measuring other parameters.

Representative examples of the pulse generator 51 may include electronic circuitry and hardware for performing audio-based communication and audio-driven commands to and from the pulse generator 51. A pulse generator 51 implanted within the upper gastrointestinal tract or airway makes it possible to use transmit such audio-driven commands, for example, voice or digitally generated audio streams, which otherwise would be substantially attenuated in conventional devices surrounded by tissues and/or fluid, to a receiver (not shown). For example, the receiver may be a microphone or other transducer. The receiver may be integrated within the pulse generator 51 and may be in communication with the electronic controller 86 for executing logic within the controller 86 and causing a response in the pulse generator 51 functioning.

Exemplary embodiments of the pulse generator 51 optionally may include one or more stimulation and/or sensing electrodes (not shown) positioned on or near the controller housing 50 for substantially communicating with the inner wall of the upper gastrointestinal tractor or airway when implanted. The housing electrode may be formed from an electrically conductive member, such as a metallic member, and in electrical communication with the electronic controller 86 within the controller housing 50, directly or by way of one or more electrical leads passing along the external surface of the casing to the one or more electrical lead sockets 90. In another example, one or more electrodes may be affixed to an anchor device and positioned to substantially communicate with the inner wall of the upper gastrointestinal tractor or airway upon implantation of the pulse generator 51. A controller electrode integrated with the controller housing 50 or an electrode affixed to an anchor device may perform any or all of the electrical stimulation and/or sensing functions described herein. In one example, the casing electrode may serve as a reference electrode when measuring electrical impedance in the cardiac region. In another example, a housing electrode affixed to a pulse generator 51 implanted within the esophagus or a primary bronchus may be used to electrically stimulate at least one of the right or left atrium.

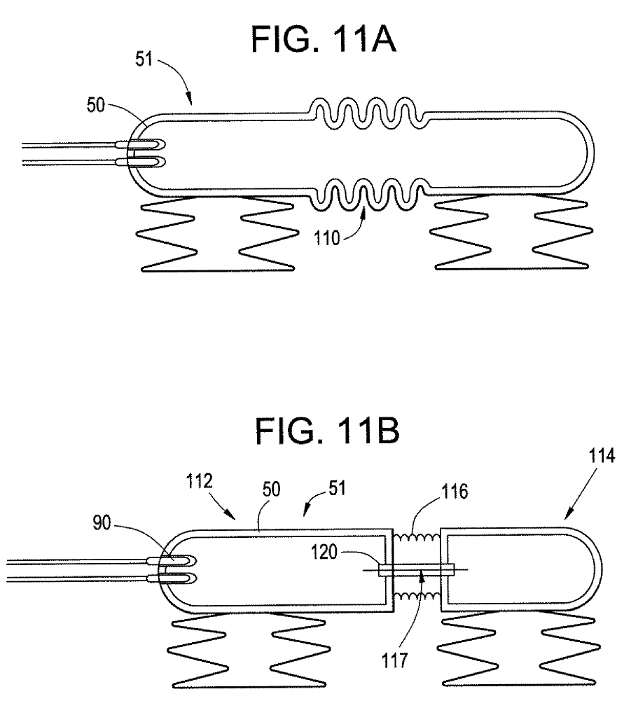

Being insertible through either the oral or nasal cavity, the controller housing 50 embodiments may be at least partially flexible to ease insertion. A flexible controller housing 50 may be constructed at least partially of elastomeric materials, for example, elastomeric polymers or polyurethane. In other embodiments, a metallic controller housing 50 may include one or more areas along its axis that may bend, flex, or otherwise be malleable. FIGS. 11A and 11B illustrate two possible embodiments of a flexible controller housing 50. The controller housing 50 illustrated by FIG. 11A includes one or more corrugated areas 110, allowing the controller housing 50 to flex or bend at the corrugated areas 110. Though not illustrated, the pulse generator 51 illustrated in FIG. 11A further includes one or more of the pulse generator elements described with reference to FIG. 8, though these elements may be designed and positioned so as to not restrict or interfere with the flexion of the controller housing 50.

FIG. 11B illustrates another example controller housing 50 configuration that also aids in longitudinal casing flexibility. This controller housing 50 embodiment includes at least two sub-cases 112, 114, each housing some of the components as described with reference to FIG. 9, and connected by one or more flexible connectors 116. Thus, the controller housing 50 may bend or otherwise flex around the flexible connectors 116, and be electrically connected by a non-rigid electrical conductor 117. The flexible connectors 116 may be constructed from metallic materials, polymeric materials, or any combination thereof and may be formed so as to limit longitudinal (or axial) movement, but permit lateral flexion at the connectors 116. In one embodiment, the flexible connectors 116 may be formed as a spring-like structure; though other configurations suitable to provide the desired flex may be used. The non-rigid electrical conductor 117 may be an insulated, or otherwise electrically isolated lead, and may provide electrical communications between components within each sub-case 112, 114. In one example, each sub-case 112, 114 may include a hermetically sealed electrical junction 120, such as a feedthrough, operable to receive an end of the non-rigid electrical conductor 117. One or more of the sub-cases 112, 114 may be separated from the controller housing 50, for example, for servicing, programming, calibration, or data abstraction. For example, in one embodiment, the proximal sub-case 114 (that opposite the case including lead sockets 90) may house a power source, which may be removable for charging or replacement, without requiring removal of the entire pulse generator 51 from the implantation site, thus avoiding disengaging leads, anchor devices, and the like.

In another possible embodiment of a controller housing implantable within an airway (not shown), a tracheal component and a bronchial component may be provided for implanting at least partially within the trachea and one of the right or left primary bronchus. A controller housing configured in this manner may be larger in size than an embodiment implanted solely within the trachea or implanted solely within the primary bronchus. Accordingly, the diameter of tracheal component may be substantially larger than the diameter of the bronchial component 119, optimizing the controller housing volume while minimizing any interference to airflow through the airway. In another embodiment, the controller housing may be implantable within the trachea and both primary bronchi. A controller housing implantable within both bronchi and the trachea may be configured in an inverse “Y” shape, and may at least partially rest at or near the bronchial bifurcation. The controller housing of these embodiments may be anchored to the trachea, the primary bronchus, or to both, by an anchor device, such as described herein.

Electrodes and Electrical Leads

The electrodes may be operable to provide electrical stimulation or to perform physiological sensing and measurement; though, in some embodiments the electrodes may be operable to perform both stimulation and sensing. An electrode generally may include an electrode body and at least one stimulation surface from which electrical signals may be delivered. The stimulation surface may be a conductor for sensing cardiac electrical activity or other cardiac activity. The electrodes may be mipolar electrodes used in cooperation with another reference electrode, or bipolar or tripolar electrodes, including both a different and indifferent pole. In particular embodiments, the electrodes are affixed or integrated with an electrical lead at or near its distal tip. However, in other embodiments, such as those including an electrical lead carrying more than one electrode, at least one electrode may be affixed to the electrical lead at a position proximal from the distal end, to allow additional stimulation or sensing at a position proximal to the distal tip of the electrical lead.

The electrodes and leads may be guided to and positioned at the desired implantation site using delivery devices, such as a catheter, a guidewire, a combination thereof, or other known means for guiding elongate devices within a body lumen.

The electrodes may be fixable at one or more selected implantation positions within the upper gastrointestinal tract or airway to prevent electrode migration from the selected position and to promote electrical coupling with the epithelial tissue lining. Various anchoring devices may fix the electrode at the selected implantation position. For example, these anchoring devices may include one or more barbs, one or more hooks, suture, staples, one or more extensible members, one or more stent-like expandable members, a balloon, an adhesive, or any combination thereof, as described herein. Barbs or hooks may be in a fixed relationship with the electrode, or may be selectably retractable by way of mechanical, electrical, chemical means, or the like. Extensible members may include, for example, members made of self expandable metals (e.g., nickel-titanium, cobalt alloy, stainless steel, shape memory alloys), members made of self expandable polymeric materials (e.g., silicone), or mechanically extensible members, such as those stent-like expandable members described with reference to FIGS. 8A-8E for use in conjunction with the controller housing 50. Electrodes implantable within a patient's bronchi may be proportioned to have a diameter slightly larger or approximately the same size as the inner bronchi at the selected implantation site, causing the electrode to lodge within the airway. Though, electrode embodiments proportioned to lodge within the airway as a result of its diameter may optionally include a lumen or passageway formed axially through the body of the electrode and in parallel with the airway lumen to permit airflow through the passageway or lumen, thus avoiding interference with respiratory activities occurring at or downstream of the implantation site.

In certain embodiments, the electrode or electrodes may be at least partially coated with an insulating material. Examples of suitable materials may include a polymer insulator (such as silicone, polyurethane, polytetrafluoroethylene (e.g., Teflon™), or other fluoropolymers), a ceramic insulator, or a glass insulator. An insulative coating may enable the control, direction, and focus of the stimulation signal sent by the pulse generator. An insulative coating may also allow one to divide the electrode into multiple electrode stimulation regions for optimizing the stimulation location and/or for operating in a multi-electrode configuration, such as a bipolar or tripolar electrode.

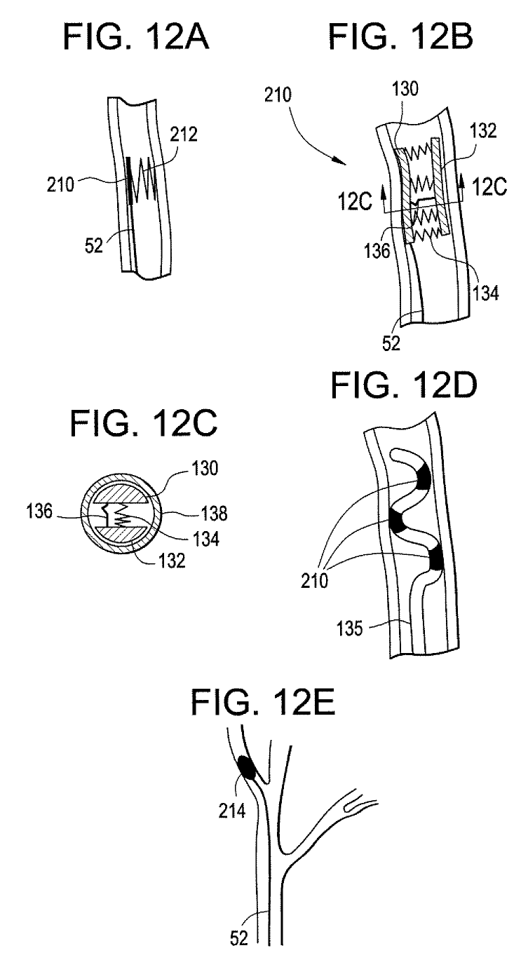

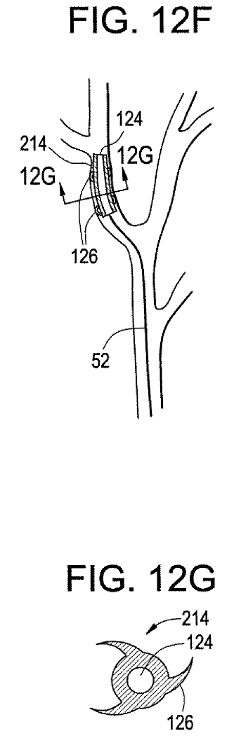

FIGS. 12A-12G illustrate several exemplary embodiments of electrode configurations that aid in fixation and retention within the upper gastrointestinal tract or the airway, improve electrical coupling. Example electrode configurations and anchor devices are similar in design and function to those described with reference to the controller housing anchor devices. Accordingly, any of the anchor device embodiments described with reference to the controller housing herein may be applied as anchor devices for an electrode, and any anchor device embodiments described with reference to the electrodes herein may be applied as anchor devices for the controller housing. However, some of the electrode anchor device embodiments described herein may only be practicable for implantation at a position within the airway, for example within the bronchi or bronchioles, because they substantially restrict flow or passage through the lumen.

FIG. 12A illustrates an electrode 210 embodiment including an anchor device 212 similar to those described with reference to FIGS. 8A-8E for use with the controller housing. The electrode 210 is carried on the end of an electrical lead 52 and positionable and fixable at a position within the esophagus, trachea, bronchus, bronchioles, or any branch thereof. In this embodiment, the anchor device 212 may be an extensible anchor device, such as a stent-like expandable member, as further described herein. Other example anchor devices 212 may include one or more radially expandable circumferential rings, balloon sleeves, radially extensible rigid members, tubular members, texturing, suture, staples, barbs, hooks, studs, adhesive, shape memory alloy members, or any combination thereof. In one embodiment, the anchor device 212 may be formed from electrically conductive material and serve as part of the electrically conductive electrode 212 function, so as to further promote electrical stimulation or sensing effectiveness.

FIGS. 12B and 12C illustrate another embodiment of electrode 210, which includes at least two electrode sub-components 130, 132 configured similar to the controller housing configuration described with reference to FIGS. 8D and 8E. The at least two electrode sub-components 130, 132 may be connected by one or more expandable connectors 134 which create opposing forces against the two electrode sub-components 130, 132, radially biasing each against the inner wall of the esophagus or airway at opposite areas. The expandable connector 134 and electrode sub-components 130, 132 fix the electrode 210 at the implantation site while leaving a passageway between the electrode sub-components 130, 132 through which gasses, solids, or fluids may pass. The expandable connector 134 may be configured similar to any of the expandable members described herein with reference to example electrode and/or controller housing embodiments. In various embodiments, the expandable connectors 134 may be formed as a metallic or polymeric radially expanding member or spring, from a shape memory alloy, as mechanically adjustable rigid members, as telescoping members, or the like. In one embodiment, the two electrode sub-components 130, 132 may further be electrically coupled by one or more isolated electrical connector 136, to facilitate electrical communication between each electrode sub-components 130, 132. In another embodiment, the one or more expandable connectors 134 may double as an isolated electrical connector 136, eliminating the need for an additional isolated electrical connector. For embodiments that are implantable within the esophagus, the expandable member is preferably a radially expanding member, similar to an esophageal stent, as described with reference to FIGS. 8D and 8E, to provide a substantially unobstructed passageway and permitting solids to pass therethrough.

As illustrated in FIG. 12C, the two or more electrode sub-components 130, 132 connected by one or more expandable connectors 134 may be compressed to a reduced profile during delivery and positioning, for example using a delivery device 138, such as a catheter. In one embodiment, the two electrode sub-components 130, 132 each have a substantially semi-circular cross section, each being complementary in shape to the other, and having an external radius of curvature substantially similar to the inner wall of the esophagus or airway in which it may be implanted. Although the embodiment illustrated by FIGS. 12B and 12C may include only two electrode sub-components, the electrode 210 may be formed from any number of electrode sub-components, each being biased radially against the inner wall at the implantation site. In another embodiment, only one of the electrode sub-components 130, 132 may be electrically conductive and serve the electrode function.

FIG. 12D illustrates another suitable electrode and electrical lead embodiment, which includes at least one electrode 210 and at least one pre-shaped electrical lead 135. The distal end of the pre-shaped electrical lead 135 may be pre-shaped, such that the shape will substantially lodge or wedge within the lumen of the upper gastrointestinal tract or airway in which it may be implanted. As illustrated in FIG. 12D, the pre-shaped electrical lead may be shaped substantially waved shape, such as an “S” shape, such that the wave amplitude may be substantially similar or slightly larger than the inner diameter of the lumen in which it may be implanted. In other embodiments, the pre-shaped electrical lead 135 may be formed in other shapes, such as a spiral, circular, elliptical, or hooked, for example. The pre-shaped electrical lead 135 may carry one or more electrodes 210. The embodiment illustrated in FIG. 12D includes three electrodes 210, positioned at or near the distal portion, and then along the pre-shaped portion of the electrical lead 135. Positioning the one or more electrodes 210 at or near a maximum or minimum of the pre-shaped form causes the electrode or electrodes 210 to be biased against the inner wall of the esophagus or airway lumen, thus increasing the electrical coupling therewith.

The pre-shaped portion of the electrical lead 135 may include a less pliable, less flexible and more shape resilient material than the remaining proximal portion of the lead 135. In one example, the pre-shaped portion of the electrical lead may be coated or otherwise constructed at least partially with polyurethane whereas the remaining proximal portion may be coated or constructed at least partially with silicone. In another example, a shape memory alloy, such as nickel titanium alloy, may be integrated with the pre-shaped portion of the electrical lead 135, such that upon application of energy, the electrical lead 135 may transition from a substantially straight shape to assume any pre-defined shape, for example an “S” shape as illustrated.

A pre-shaped electrical lead 135 carrying one or more electrodes 210 may be delivered using a delivery device, such as a catheter, sheath, stylet, or guidewire. Accordingly, when contained within a lumen of the delivery device, the pre-shaped electrical lead 135 may be substantially straightened for delivery and positioning at or near the implantation site. Upon removing the delivery device, the pre-shaped portion of the electrical lead 135 may reform to it's pre-shaped form, causing it to apply a force against the wall of the esophagus or airway lumen and substantially affix the electrode 210 and electrical lead 135 in place.

FIG. 12E illustrates an electrode 214 positioned at the distal tip of an electrical lead 52 and implantable within the airway of a patient, for example, within the secondary or tertiary bronchus, the bronchioles, or any branch thereof. The diameter of the electrode 214 is substantially similar or slightly larger than the inner diameter of the airway at the selected implantation site. For example, an electrode 214 proportioned for placement at an implantation site in the tertiary bronchus may have a diameter of approximately 5 mm to approximately 8 mm if the inner diameter of the tertiary bronchus is approximately 5 mm. Accordingly, in some embodiments, the diameter of an electrode 214 may be 0 mm to approximately 8 mm greater than the inner diameter of the airway at the implantation site. The inner diameter of a patient's airway varies depending upon the location within the airway and upon the patient; thus, the diameter of an electrode 214 in accordance with the embodiment illustrated in FIG. 12E also will vary depending upon the intended implantation site dimensions. An electrode 214 having a diameter substantially similar or slightly greater than airway lumen in which it is implanted will aid electrode 214 retention at the implantation site and promote electrical coupling for more effective stimulation or sensing. Additional anchor devices, such as texturing, suture, staples, barbs, hooks, studs, adhesive, shape memory alloy members, or any combination thereof, optionally may be included to enhance electrode 214 retention in the airway. Accordingly, for certain electrode embodiments that include anchor devices, the electrode diameter need not be the same or slightly larger as the airway, but may optionally be a slightly smaller diameter than the airway.

The embodiment illustrated in FIG. 12E, without further design enhancements, may inhibit airflow downstream from the implantation site. Accordingly, this embodiment may be generally suited for implantation sites located in the periphery of the bronchi, having relatively smaller diameters such that the restricted airflow passage may be clinically insignificant, for example, within smaller branches of the tertiary bronchi or within the bronchioles. Furthermore, an electrode of this embodiment cannot be implanted within the upper gastrointestinal tract of a patient without substantially obstructing the passageway, rendering this design impractical for esophageal implantation.

FIG. 12F illustrates another example electrode 214 including at least one a lumen or passageway 124 extending axially through the electrode 214 to permit airflow through therethrough. The lumen 124 may extend axially from the electrode's 214 proximal end to its distal end, and may be centered radially or offset from the central axis of the electrode 214. The lumen 124 may be proportioned to permit substantial airflow to pass therethrough. The lumen 124 may further provide a passageway with which delivery devices may cooperate, such as a guidewire, a bronchoscope, or the like for delivering and/or securing the electrode at the implantation site. An offset lumen 124 leaves greater room thin the electrode 214 body for electrical circuitry or sensing components as may be called for. In other similar electrode embodiments, a recess may be formed in one or more external surfaces of the electrode 214 and extend from the proximal end to the distal end (not shown). When implanted in the airway, an electrode 214 having a recess leaves a passageway through which air may pass between the recess and the inner wall of the airway. In addition to the lumen 124 or recess, the electrode 214 body may include one or more protrusions 126, such as studs, barbs, hooks, shape memory alloy members, or any combination thereof, extending substantially radially from the electrode 214 body for engaging the inner wall of the airway to aid in electrode 122 fixation. Furthermore, one or more of the protrusions 126 may be formed from the electrically conducting material of the electrode 214 to promote electrical coupling by improving surface area contact with the inner wall of the airway.

FIG. 12G illustrates a cross-section of the electrode 214 embodiment illustrated in FIG. 12F. Accordingly, this embodiment may include a lumen 124 passing through the electrode 214 body and multiple protrusions 126 extending from the body. As illustrated in FIG. 12G, the protrusions 126 may have a hooked shape, though other shapes may be employed, such as pointed, barbed, rounded, and the like. The protrusions 126 illustrated in FIGS. 12F and 12G are equally applicable to other electrode embodiments described herein. An electrode 214 according to the embodiment illustrated in FIGS. 12F and 12G may be dimensioned for placement within the esophagus, as well, such that the inner lumen 124 has a large enough diameter to substantially permit solids to pass therethrough.

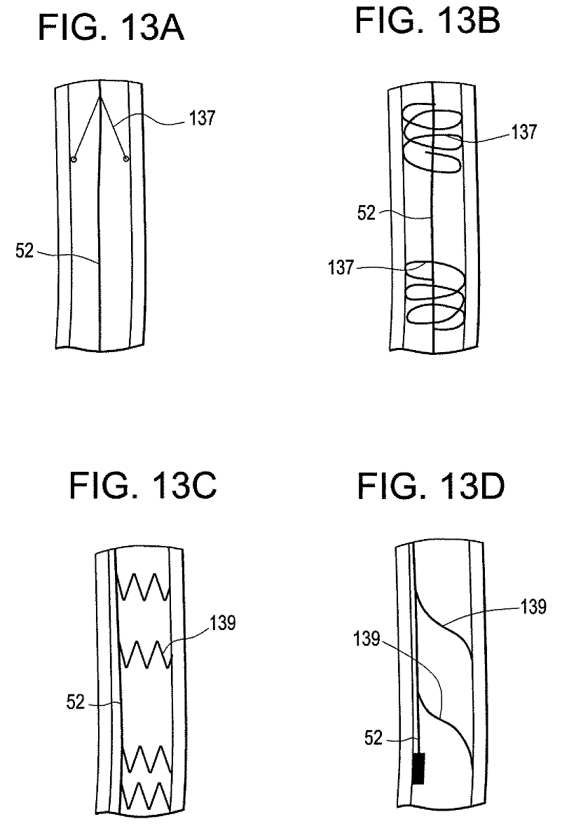

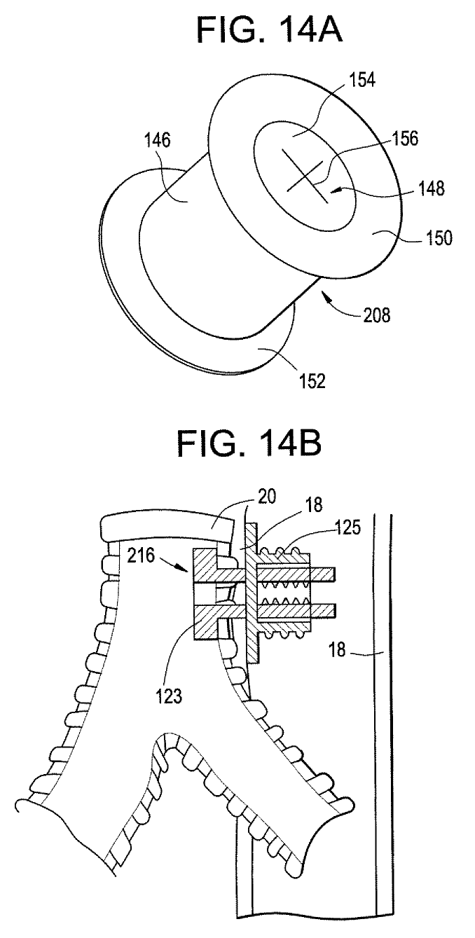

FIGS. 13A-13D illustrate some embodiments of electrical leads 52 that include at least one lead securing member for substantially retaining the electrical lead 52 at a certain position within the upper gastrointestinal tract, such as the esophagus, or within the airway, such as the trachea or bronchus. Because the electrical lead 52 is a foreign object to the tissues, irritation may occur, causing discomfort and/or other undesirable conditions, such as chronic coughing, itching, or tissue granulation. In one embodiment, as illustrated in FIG. 13A, at least two lead securing members 137 may be formed as a pin, arm, rod, or other member extending radially from the electrical lead 52 in approximately opposite directions and substantially affixing to or exerting pressure against the inner wall of the gastrointestinal tract or airway in which the electrical lead 52 may be positioned, and the electrical lead 52 is suspended therebetween. In another embodiment, as illustrated in FIG. 13B, the lead securing member 137 may be formed as a coil or other radially extending elliptical member, such that the lead securing member is in at least partial contact around the circumference of the inner wall of the esophagus or airway and the electrical lead 52 is suspended therebetween. The lead securing members 137 illustrated in FIGS. 13A and 13B allow the electrical lead 52 to be suspended within the interior lumen of the esophagus or the airway, and avoid substantial contact with the inner wall.

FIGS. 13C and 13D illustrate still other embodiments of a lead securing member 139 which biases the electrical lead 52 toward the inner wall of the gastrointestinal tract, such as the esophagus, or of the airway, such as the trachea or bronchus. In this way, the electrical lead 52 may be at least partially or totally encapsulated by the epithelial tissue of the inner wall, so as to allow the body's natural mechanisms to protect against and combat contamination, such as bacteria or infection resulting therefrom. In one embodiment as illustrated in FIG. 13C, the lead securing member 139 may be an expandable member, similar to those described with reference to FIG. 8A or 12A, that radially exerts a biasing force against the inner wall of the esophagus or airway and causes the electrical lead 52 to interface with the inner wall opposite the lead securing mechanism 139. In this example, the lead securing member 139 may be configured as a radially expandable member, such as a stent-like member, an inflatable balloon sleeve, a spring, or a coil. In another embodiment, as illustrated in FIG. 13D, the lead securing member 139 may be configured as a pin, arm, rod, or other member extending radially from the electrical lead 52 in an approximately opposite direction and substantially affixing to or exerting pressure against the inner wall of the esophagus or airway opposite the electrical lead 52, creating a biasing force which causes the electrical lead 52 to interface with the inner wall opposite the lead securing member 139. In still another embodiment, the lead securing member 139 may be like one of the electrode anchor devices or controller housing anchor devices described herein and illustrated in FIG. 8 or 12. The lead securing members 137, 139, as described herein, may be formed of a biocompatible elastomeric material or shape memory material. Examples of these materials include elastomeric polymers (such as silicone, polyurethane), flexible metals, and shape memory alloys (e.g., Nitinol™).

The electrical leads carrying one or more electrodes may be of any known design, including unipolar, bipolar, tripolar, multi lumen, single lumen, coaxial, or bifurcated. The electrical lead may be insulated, for example by silicone, polyurethane, silicone with polyurethane overlay, or any other material known in the art to be suitable for electrically isolating medical leads.

In some embodiments, the electrical lead may have a variable length. For example, it may be longitudinally extensible and retractable to aid in delivery and implantation of the electrode or the pulse generator. As another example, the electrical lead may be configured as an expandable and retractable coil, in a telescoping configuration, or the like. The ability to change the electrical lead length may facilitate implanting the one or more electrodes and the pulse generator. In other embodiments, however, the electrical leads may not be independently variable, but may be adjusted when securing to the pulse generator during implantation.

The electrical leads optionally may include a radiopaque coating or a radiopaque material to aid in delivery when using imaging techniques, such as x-ray, computed tomography, or fluoroscopy, for example. Furthermore, example electrical leads may be capable of eluting and/or delivering medicinal agents to reduce rejection of the lead and electrode by the surrounding tissue, therefrom. For example, the electrical leads may be coated with steroids, anti-inflammatory agents, anti-bacterial agents, antibiotics, or any combination thereof, as are known. In other embodiments, the electrical lead may include a lumen existing therethrough for selectively delivery of such medicinal agents, for example, during electrode delivery as administered by the physician, or while implanted as released from the pulse generator or other source.

Cannula

In one embodiment, the esophagus and the trachea may be penetrated and one or more apertures may be formed therethrough for passing at least one electrical lead carrying at least one electrode between the esophagus and the trachea. In one embodiment including multiple implantable electrical leads, an aperture for each electrical lead may be formed in the esophagus and the trachea, to reduce the aperture sizes and to reduce the friction caused within each aperture to minimize stresses caused on the electrical lead or on the tissue walls. A cannula may optionally be implanted in the walls of the esophagus and the trachea, such as is illustrated in FIG. 7C, to aid in sealing the thoracic cavity, the esophagus, and the airway, to exclude the passage of air, biological contaminants, or biological fluids therebetween, to provide structural integrity to the aperture in the tissue wall, to house the electrical leads to ease movement therethrough, and to reduce irritation, inflammation, or infection where the electrical leads may otherwise contact the esophagus or trachea wall. In some embodiments, however, the cannula may not be implanted in the esophagus and trachea walls, but may be affixed to the inner or exterior walls of the esophagus and trachea and around the apertures formed therein. The cannula may be formed in any shape suitable to be implanted in the esophagus and trachea and to permit one or more electrical leads to pass therethrough, such as tubular, sleeve-like, disk-like, or elliptical, for example. The cannula may be constructed from any biocompatible materials suitable for subcutaneous implantation and to provide at least partial rigidity and structural support in the passage, such as metals, polymers, or any combination thereof. The cannula optionally may be at least partially coated by anti-inflammatory or anti-bacterial agents or materials, such as an antibiotic, steroid, or silver metal coating. Furthermore, in an embodiment having multiple apertures formed in the airway wall, cannulae may be dimensioned to position an individual cannula in each aperture.

FIG. 14A illustrates one example of a cannula useful with the systems and methods described herein. The cannula 208 may be formed as a sleeve or conduit, having an outer surface 146 and an inner surface (not shown) existing within the cannula 208, and defining an orifice 148 extending therethrough. In one embodiment, the inner diameter of the cannula 208 may range from approximately 1 mm to approximately 4 mm, to allow for passing one or more electrodes therethrough. In another embodiment, the cannula 208 may have multiple orifices for passing individual leads therethrough, such that each individual orifice may have a diameter of approximately 1 mm to approximately 4 mm. In one embodiment, the length of the cannula 208 may range from approximately 2 mm to approximately 15 mm, which may generally depend upon the configuration of the cannula 208 and the actual esophagus wall and trachea wall thicknesses; although, the cannula 208 dimensions may depend upon the size of the passage, which may ultimately depend, for example, upon its position, the size of the patient, or the number of electrical leads to pass therethrough. The cannula 208 optionally may include a first flange 150 and a second flange 152 extending radially from opposite ends of the cannula 208. The first and second flanges 150, 152 are positionable against the inner wall of the esophagus and the inner wall of the trachea to aid in retaining the cannula 208 implanted in the aperture and to aid in sealing the thoracic cavity from the esophagus and the airway environments. The first and second flanges 150, 152 may further have a preformed curvature approximating that of the curved surfaces of the inner esophagus and the inner trachea, respectively, to aid in sealing, retention, and/or comfort. In one embodiment, additional flanges may be formed on the cannula 208 extending radially from the cannula 208 and dimensioned such that they are positionable against the outer wall of the esophagus and the outer wall of the trachea.

In various embodiments, the cannula 208 may further include an inner membrane 154 extending between at least one of the flanges 150, 152 and across the orifice 148, having one or more slots or apertures 156 formed therethrough. The aperture 156 may be dimensioned to have approximately the same or slightly smaller diameter as the electrical lead or leads intended to pass therethrough, such that the aperture 156 forms at least a partial seal around the electrical lead or leads. The inner membrane 154 allows passage of the electrical leads and provides further isolation between the environments. The inner membrane 154 may be formed from any biocompatible elastomeric material suitable for subcutaneous implantation, such as elastomeric polymers, for example. Though not shown, two inner membranes 154 may be included, one on each end of the cannula and extending between each flange 150, 152.

The cannula 208 may be formed from pliable materials, for example, elastomeric polymers, such as silicone or polyurethane, such that they may be at least partially compressed within a lumen of a delivery device, such as a catheter or other lumen. When properly positioned and upon release from the delivery device, a pliable cannula 208 may expand into place in the apertures formed in the esophagus and the trachea and each of the flanges 150, 152 may expand radially inside the esophagus and the trachea, respectively. Various other cannula designs and shapes are envisioned, and any cannula suitable for the functions described herein may be used.

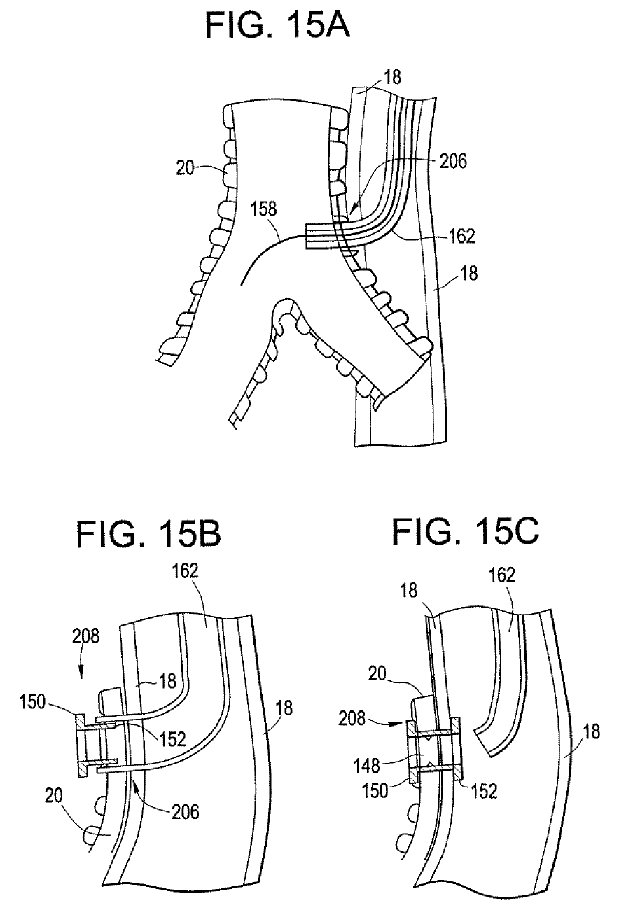

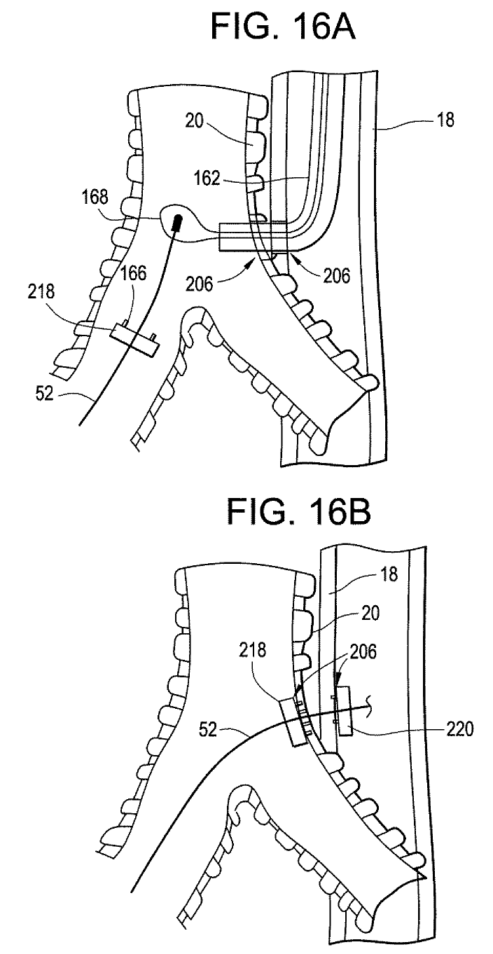

FIG. 14B illustrates another embodiment of a cannula useful in the systems and methods described herein. Cannula 216 may be configured in a manner similar to that illustrated by FIG. 14A but including two interconnecting sleeves (or interconnecting flanges), an airway sleeve 123 adapted for implantation in the trachea from the interior and a gastrointestinal sleeve 125 adapted for implantation in the esophagus from the interior. In one example, the airway sleeve 123 may have an exterior diameter substantially the same or slightly smaller than the inner diameter of the gastrointestinal sleeve 125 to allow for slidably connecting them. Each of the airway sleeve 123 and gastrointestinal sleeve 125 may have flanges extending radially therefrom, one or more orifices extending therethrough, and/or one or more membranes or sealing rings, as described with reference to FIG. 14A. Slideably connectable sleeves 123, 125 forming a cannula 216 may adjustably compensate for esophagus and trachea wall thicknesses, thus improving the seal formed by the cannula 216. A cannula 216 configured in this manner may be implanted in the aperture in manners similar to those described herein with reference to FIG. 15A-15C or 16A-16B.

In other embodiments, two cannulae, such as the cannula 208 illustrated in FIG. 14A or the cannula 216 illustrated in FIG. 14B, may be used, one implantable within the wall of the esophagus and one implantable within the wall of the trachea. In embodiments using two cannulae, the esophagus and trachea are not as mechanically coupled and thus less restricted, than in embodiments including a single cannula, such as in FIG. 14A or 14B.

FIGS. 15A-15C illustrate a cross section of one embodiment of a cannula 208 that may optionally be implanted within the wall of the esophagus and the trachea through apertures 206, and represent exemplary stages in one method for implanting the cannula 208. FIG. 15A illustrates an initial stage during the implantation of the cannula 208 within the esophagus and trachea. The cannula 208 may be implanted during, or subsequent to, forming an aperture 206 or passage in the esophagus 18 and in the trachea 20, such as by using a needle or wire 158, or a cutting device, such as a blade or scissors. In some embodiments, the apertures 206 may be formed with the wire 158 through the wall of the esophagus 18 and between the cartilage rings 160 of the trachea 20. If necessary or desired, the diameter of the apertures 206 may be increased using a fenestrator, catheter tip, blade, scissors, needle, or wire 158, or other suitable device for forming and/or opening an aperture in a tissue lumen wall. Subsequent to opening the apertures 206 to the desired size, a cannula delivery device 162, such as a catheter or other elongated lumen for delivery, may be inserted through the patient's oral or nasal cavities, into the esophagus 18, though the aperture 206 formed in the esophagus, and advanced through the aperture 206 formed in the trachea 20 into the airway. The cannula delivery device 162 may be inserted through the apertures 206 over the needle or wire 158, such as if using a guidewire, or the needle or wire 158 may be removed prior to insertion of the cannula delivery device 162.

FIG. 15B illustrates a cross section of the cannula 208 during another stage of the implantation method. In this embodiment, the cannula 208 may formed from pliable materials and compressed within the delivery device 162 for delivery through the esophagus 18 and the trachea 20. FIG. 15B illustrates the cannula 208 partially disposed within the distal tip of the delivery device 162 and partially released such that the first flange 150 is expanded radially within the airway of the trachea 20 and the second flange 152 remains compressed within the delivery device 162.

FIG. 15C illustrates a cross section of the cannula 208 implanted in the esophagus 18 and the trachea 20 walls after removing the delivery device 162. Properly implanted, the first flange 150 is positioned substantially against the inner wall of the trachea 20 and the second flange 152 is positioned substantially against the inner wall of the esophagus. Accordingly, the first and second flanges 150, 152 serve to retain the cannula 208 in the esophagus and trachea walls, as well as provide an additional barrier between the environments (e.g., sterile, non-sterile). In one embodiment, the cannula 208 may include an anchor device for retaining the cannula 208 in place, similar to certain devices described herein with reference to certain controller housing or electrode embodiments, such as one or more hooks, barbs, studs, suture, staples, or adhesive.

Upon implanting the cannula 208 in the trachea wall, one or more electrical leads may be passed into the patient's airway from the controller housing implanted in the esophagus, through the cannula orifice 148. Alternatively, the electrical lead may be orally or nasally inserted into the patient's airway, as described with reference to other embodiments herein, and may be passed from within the airway, through the cannula 208, and to the esophagus implanted controller housing. In other embodiments, however, one or more electrical leads may be pre-inserted into the cannula and carried through the esophagus 18 and trachea 20 concurrent with implanting the cannula 208. In another embodiment, the electrical lead may be implanted through the apertures formed in the esophagus 18 and the trachea 20 and the cannula 208 may be subsequently passed over the electrical lead for implantation. In embodiments including two cannulae, one for implantation within the esophagus wall and one for implantation within the trachea wall, the above-discussed cannula implantation method may be performed for each cannula.

FIGS. 16A and 16B illustrate a cross section of one embodiment of a cannula that optionally may be affixed to the inner or exterior wall of the esophagus or the trachea around apertures formed therein, and represent exemplary stages in a method for implanting the cannula. FIG. 16A illustrates a cannula 218 integrated with an electrical lead 52, such that the electrical lead 52 passes through the cannula 218. This cannula 218 may be formed in a disk-like shape having an orifice extending therethrough. The cannula 218 may serve as a flange for mounting or affixing to the inner or outer walls of the esophagus 18 and trachea 20 around the apertures 206. Similar to the cannula described with reference to FIG. 14A, an inner membrane may also extend across the orifice for retaining the electrical lead or leads and to provide an additional seal. The electrical lead 52 may slide within the cannula 218 to allow for adjustment and freedom of movement during positioning and implantation of the electrode, the controller housing, and the cannula. The cannula 218 may further include one or more anchor devices 166, similar or identical to other anchor devices described herein. At least two cannulae 218 may be used for implantation, one around the aperture 206 formed in the esophagus wall and one around the aperture 206 formed in the trachea wall. The same methods for implanting the cannula 218 on the inner walls may be used to implant the cannula 218 on the exterior walls of the esophagus or trachea.

The electrical lead 52 may first be orally or nasally inserted into the patient's airway having the cannula 218 thereon. As illustrated in FIG. 16A, a delivery device or lumen 162 may be passed from the esophagus through the apertures formed in the esophagus and trachea walls into the airway. A retrieval tool 168, such as a lasso, snare, forceps, hook and eye, or the like, may be passed through a lumen of the delivery device 162 into the airway. The retrieval tool 168 is adapted to grasp the proximal end of the electrical lead 52 and pull it through into the delivery device 162 lumen. After receiving the proximal end of the electrical lead 52, the delivery device 162 may be pulled through the aperture in the trachea 20 until the cannula 218 affixes to the trachea's 20 inner wall. Affixed to the inner wall, optionally by one or more anchor devices 166, the cannula 218 retains the one or more electrodes passing therethrough, as well as substantially seals the aperture formed in the trachea, excluding passage of air or biological fluids between the airway and the thoracic cavity. Similarly, another cannula 220 may be passed over the electrical lead in the esophagus 18 for affixing to the esophagus's inner wall, retaining the electrical lead and substantially sealing the aperture.

FIG. 16B illustrates the cannula 218 anchored to the inner wall of the trachea 20 and the cannula 220 anchored to the inner wall of the esophagus 18 by anchor devices 166. As is shown, the electrical lead passes from within the airway, through the cannula 218, and to an esophagus implanted controller housing. In another embodiment, the cannula 218 and the cannula 220 may be affixed to the exterior wall of the trachea and the esophagus, respectively, and the electrical lead 52 and electrode may be implanted by passing the delivery device 162 through the cannula 218 and into the airway, and passing the electrical lead 52 therethrough to the selected implantation position within the bronchi.

Cannula designs and methods other than those described herein may be used to aid in the retention of electrical leads and sealing the thoracic cavity from the gastrointestinal tract and the airway. For example, certain embodiments may not include a cannula, but may allow the one or more electrical leads to pass directly through the apertures formed in the esophagus and the trachea walls. In other embodiments, other techniques and materials, such as an adhesive, a membrane, suturing, or stapling, may be used for sealing the apertures.

In some embodiments in which one or more devices are passed between the esophagus and the trachea, through the thoracic cavity, contamination from within the gastrointestinal tract or the airway may be prevented and/or treated to promote a more sterile environment. For example, in some embodiments, the electrode, electrical lead, cannula, or other device may be covered with a sterile sleeve prior to passage from the esophagus or airway. In other embodiments, the electrode, electrical lead, cannula, or other device may be treated (e.g., coated) with an antimicrobial material, such as antiseptic and/or antibiotic agent. Furthermore, the patient may be treated with antibiotics, steroids, or other pharmaceutical agents systemically or by inhalation, prior to and/or after the implantation procedure.

Wireless Tissue Interface

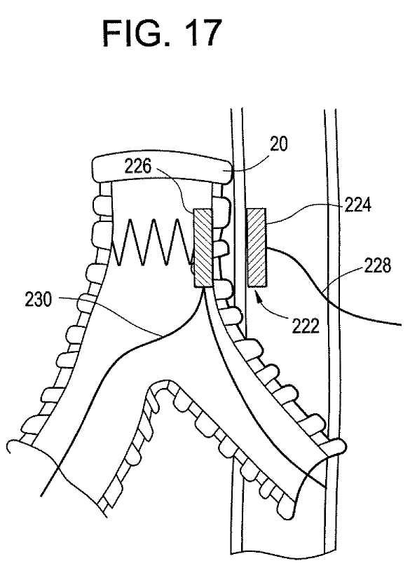

In one embodiment, as described herein, the system may include a tissue interface adaptable for wirelessly communicating between a pulse generator implantable within the upper gastrointestinal tract and electrodes implantable within the airway, rather than forming apertures in the esophagus and trachea. FIG. 17 illustrates a cross section of an example tissue interface 222 operable for wireless communication, according to one embodiment. In one example, the tissue interface 222 may be formed as two components: an gastrointestinal interface 224 and an airway interface 226. The esophagus interface 224 is adaptable to couple with one or more gastrointestinal lead portions 228 attachable to a controller housing implantable within an upper gastrointestinal tract such as the esophagus 18. The gastrointestinal lead portion 228 is operable to wirelessly communicate electrical signals to and from one or more airway lead portions 230 positioned within the airway. Accordingly, the airway lead portion 230 is adaptable to couple to the airway interface 226 at its proximal end. In another embodiment, the gastrointestinal interface 224 and the airway interface 226 may be integrated with the distal end of the gastrointestinal lead portion 228 and the proximal end of the airway lead portion 230, respectively. The lead portions 228, 230 and the tissue interface 222 may be implanted by any implantation methods described herein.

The airway interface 226 and the gastrointestinal interface 224 may be affixed to the inner walls of the airway 20 and the esophagus 18, respectively, by anchoring devices similar to certain devices described herein with reference to the controller housing or electrode embodiments. For example, the anchoring device may utilize one or more hooks, barbs, studs, suture, staples, or adhesive. In another embodiment, the airway interface 226 and the gastrointestinal interface 224 may be affixed to the respective inner walls by magnetic fixation, such as by integrating or affixing polar opposite magnets to the airway interface 226 and the gastrointestinal interface 224.

Electrical signals may be wirelessly communicated across the tissue interface 222 by electromagnetic induction, for example. Alternatively or in addition, other wireless means for transmitting electrical signals may be employed, such as radio frequency, ultrasonic, infrared, or other electromagnetic waves. For example, the airway interface 226 and the gastrointestinal interface 224 may each have a wireless transmitter and receiver operable to communicate wirelessly through protocol, such as radio frequency, microwave, infrared, for example. The airway interface 226 may include electronic circuitry, a power source, hardware, and/or software for receiving and transmitting wireless communications from and to the pulse generator, and for generating electrical stimulation pulses or performing sensing functions. Thus, electrical stimulation or sensing functions may be divided, with at least some of the electrical stimulation signals being generated within the airway, for example within the airway interface 226, and at least some of the logic for determining timing, delay, magnitude, and the like of signals occurring within the pulse generator implanted within the gastrointestinal tract. Furthermore, at least part of the sensing functions may be performed within the airway and communicated wirelessly to through the tissue interface 222 to the pulse generator.

In another embodiment, the airway interface 226 need not include a power source. For example, the energy required to operate the device may be transmitted through the tissue interface 222, for example, like an electrical transformer including a primary coil in the gastrointestinal interface 224 and a secondary coil in the airway interface 226. Generating an oscillating current in the primary coil will then induce a current in the secondary coil, as is known. In certain embodiments having a primary and secondary coil, the current may be coded to allow communicating information in the current, such as signals or commands to the airway interface 226. In one embodiment, the airway interface 226 may include electronic circuitry for receiving the current, optionally decoding the information transmitted thereby, and for generating electrical signals, such as for stimulation or sensing.

In other embodiments, the electronic circuitry for performing stimulation and/or sensing may be integrated within or near the electrode carried by the airway lead 230. In yet other embodiments, at least one or both of the gastrointestinal lead 228 or the airway lead 230 may be unnecessary. Instead, wireless communications may be transmitted directly from the pulse generator implanted within the esophagus (or implanted within the trachea or bronchus) to one or more electrodes implanted within the airway that includes electronic circuitry, a power source, hardware, and/or software for receiving and transmitting wireless communications and for generating electrical stimulation pulses and/or performing sensing functions.

Although these embodiments including wireless communication through a tissue interface are described with a pulse generator implantable within the upper gastrointestinal tract, the pulse generator may also be implantable within the airway or subcutaneously, and communicate wireless to electrodes within the gastrointestinal tract in the manner described.

Sensing Function

As described with reference to various embodiments, the implantable cardiac stimulation system may be operable to perform sensing functions as well as electrical stimulation. Physiologic electrical activity, such as electrical potential, impedance, or other physiological parameters of the heart and/or lungs for example, may be measured by the implantable cardiac stimulation system. Electrical activity, such as is sensed using conventional electrocardiogram sensing devices, including implantable cardiac rhythm management devices, may be measured in accordance with various embodiments described herein. Sensing electrode placement within the upper gastrointestinal tract or airway and more proximate to either of the heart's ventricles or atria, such as proximate to the left ventricle or proximate to the left atria, provides the ability to better sense electrical cardiac activity therein and to better determine whether the source of any arrhythmias, such as atrial fibrillation or ventricle fibrillation, or other irregularities occur in the atrium or the ventricle. Conventional electrocardiogram devices are limited in accurately detecting the exact chamber in which irregularities occur due to their distant electrode placement, which may lead to delivery of improper treatment, such as unnecessary defibrillation treatment or misdirected signals. The ability to place electrodes within the upper gastrointestinal tract or the airway provides more proximate placement to the desired cardiac chamber, more accurate sensing, and thus improved diagnosis of cardiac irregularities and more effective, safe treatment. In one embodiment, one or more electrodes and the pulse generator may be operable to perform the physiological electrical activity sensing, in addition to or instead of electrical stimulation described herein. In another embodiment, the system may include one or more sensors operable for performing mechanical measurements, such as flow, pressure, temperature, acceleration, or strain, for performing optical measurements, such as imaging, absorption, or fluorescence, or for performing ultrasonic imaging, or any combination thereof. Furthermore, one electrode may be operable to perform both sensing and electrical stimulation functions, thus reducing the number of electrodes and electrical leads implanted within the gastrointestinal tract or the airway.

In some embodiments, at least one electrode operable for sensing may be positioned away from the heart to serve as a counter electrode for measuring cardiac electrical activity, such as electrical impedance, between one or more other electrodes implanted in various positions within the upper gastrointestinal tract or the airway. In another embodiment, ventricular tachycardia may be detected by monitoring electrical activity of the left ventricle or alternatively the right ventricle.

In another embodiment, electrical impedance may be measured across a substantial area of the lungs due to the various sensor electrode implantation sites available within the upper gastrointestinal tract and the airway, such as those locations illustrated in FIGS. 2-5. For example, electrical impedance may be measured between a sensing electrode implanted within the esophagus and a sensing electrode implanted within the bronchi or bronchioles. In another example, impedance may be measured between two sensing electrodes implanted within the bronchi, such as between the tertiary bronchi or bronchioles, of a single lung, or between the tertiary bronchi or bronchioles of the left lung and the tertiary bronchi or bronchioles of the right lung. Implanting sensing electrodes within the upper gastrointestinal tract, such as the esophagus, or the airway focuses electrical impedance measurements across the heart, may be achieved through minimal or no contribution from external devices, and thus provides more accurate measurements than from conventional systems having electrodes implanted outside of the upper gastrointestinal tract and the airway. However, in other embodiments, one sensing electrode may be implantable within the upper gastrointestinal tract or airway while a reference electrode may be positionable outside of upper gastrointestinal tract and the airway, such as an electrode implantable subcutaneously, or an epidermally placeable electrode (e.g., on the skin of the upper torso). Measuring electrical impedance in the lungs, as can be achieved in certain embodiments, can correlate to the amount of fluids accumulated within a patient's lungs, which may be used to for early detection of congestive heart failure and decompensation resulting therefrom, or for detection of other diseases or conditions that may affect electrical impedance across the lungs or other areas within the thoracic cavity.

In other embodiments in which the pulse generator or the anchor device includes an electrode, the electrical impedance may be measured between one or more other implanted electrodes and the pulse generator or anchor device electrode. For example, a cardiac device having a pulse generator electrode implanted within the esophagus and at least one electrode implanted within a tertiary bronchus or a bronchiole may provide electrical impedance measurements from between the two electrodes and thus across a substantial portion of the lungs or the heart.

In addition to monitoring cardiac electrical activity, one or more other sensors may be carried by an electrical lead for sensing mechanical activity of the heart or the lungs. For example, one or more sensors, such as an accelerometer, a strain gauge, a pressure transducer, or other sensors suitable for measuring position or movement, located within the esophagus or the bronchi in close proximity to the heart, may sense movements resulting from various sources, including lung movement during breathing, peritoneal diaphragm movement, and cardiac contractility. Because the lungs are mechanically coupled to the heart, cardiac movement, such as cardiac contractility, may be measured by sensing lung movement by one or more electrodes implanted within the bronchi. Lung movement caused by breathing is characterized by relative slow acceleration compared to cardiac contraction and may be filtered out of the measurements through signal processing, such as filtering, to isolate cardiac movement. The signal processing may be performed by the pulse generator or other electrical circuitry existing within the controller housing or external to the patient. Measuring cardiac movement may be useful for detection of atrial fibrillation, ventricular fibrillation, bradycardia, or myocardial infraction, for example. Furthermore, measuring cardiac movement can help detect uncoordinated motion of the heart chambers (e.g., the ventricles) during pacing or other electrical stimulation therapy.

In some embodiments, a feedback loop may be applied by the pulse generator between the sensed signal received by the controller that represents mechanical movement and the generation of electrical stimulation signal to the same or other electrodes implanted within the upper gastrointestinal tract or the airway. A feedback loop may provide increased control over cardiac contractility synchronization by the implantable cardiac stimulation system. For example, certain operating parameters may further control synchronization, such as the delay between the left and right side contraction of the heart, the delay between atrial and ventricular contraction, synchronization of a ventricle by applying more than one electrical stimulation to more than one area on the ventricle, optimizing the stimulation signal, such as the amplitude, width, or shape, or selecting excitation and counter electrode configurations and positions.

By delivering electrical leads through the gastrointestinal tract or the airway, access and proximity is provided to other systems, such as the aorta, the pulmonary vein, the pulmonary artery, the diaphragm, or the phrenic nerve, which are otherwise primarily accessible only through complex, invasive procedures like subcutaneous or intravascular delivery. Accordingly, other physiological parameters, such as cardiac output, blood flow, or blood pressure, for example, may be sensed, or other therapy may be provided, such as respiratory paralysis, using electrical leads delivered through the patient's gastrointestinal tract or airway as generally described herein.

In one embodiment, an electrical lead carrying an ultrasonic sensor may be acoustically coupled to one or more of the aorta, pulmonary vein, or pulmonary artery by positioning within the esophagus in close proximity thereto. In other embodiments, such as is described with reference to FIG. 6, a sensor may be carried by an electrical lead delivered through the esophagus and may further be invasively implanted within a cavity of pericardium, the heart epicardium, the cardiac muscle, or the heart chambers, for example, by penetrating the wall of the esophagus and physically implanting the sensor within the tissue. In one embodiment, the distal tip of the electrical lead or sensor may include a needle or probe to pierce the esophagus wall and secure the sensor in the tissue. However, in other embodiments, the electrical lead may be secured at least partially in the esophagus by anchoring means as described herein while the needle or probe pierces the esophagus wall and extends into the tissue. As described in reference to other embodiments, the electrical lead and sensor may be guided to the implantation site using imaging techniques, such as fluoroscopy, computed tomography, magnetic resonance imaging, x-ray, ultrasound, position emission tomography, as are known. In some embodiments, the needle or probe tip may include one or more sensors for sensing parameters, such as cardiac electrical activity, cardiac contractility, blood pressure, blood flow, cardiac motion, oxygen, or the like. A needle or probe tip may further or alternatively include an electrical stimulation electrode operable for providing electrical stimulation therapy as described herein. In one embodiment, the needle or probe tip for piercing the airway may have a relatively small diameter, such as approximately 0.1 mm to approximately 4 mm, and in some embodiments less than approximately 2.5 mm, to reduce the risks of pneumothorax, which may result from air or gas accumulating in the pleural cavity. One or more sensing electrodes may be passed from within the airway to other tissues or organs, for example into lung tissue, in a manner similar to that as described for passing from within the esophagus.

In another embodiment, the upper gastrointestinal tract or airway may be used to position one or more electrodes for stimulating systems or organs such as the upper digestive system, diaphragm, or phrenic nerve. For example, the phrenic nerve may be stimulated to activate the diaphragm or the diaphragm may be directly stimulated, to provide therapy to patient's suffering from respiratory paralysis (for example due to a lesion in the central nervous system or in the phrenic nerve). Because the phrenic nerve runs from the neck to the diaphragm, and is in substantially close proximity to the esophagus and the lungs, implanting electrodes within the esophagus or the airway provides close access thereto. Furthermore, electrodes implantable within the lower branches of the bronchus also provides close access to the diaphragm. Accordingly, a system including one or more electrodes implantable within the esophagus or airway and in close proximity to the phrenic nerve and/or the diaphragm may be operable to deliver electrical stimulation to perform diaphragm pacing. In another embodiment, one or more electrodes may be implantable at a point within the patient's upper gastrointestinal tract or airway to stimulate the patient's upper gastric system, such as the esophagus or stomach, for treating eating or digestive disorders. Because the gastric system is controlled by the sympathetic and parasympathetic nervous systems, the airway or the gastrointestinal tract may provide access to stimulation locations, such as stimulating nerves near the neck from the trachea or primary bronchi, or stimulating nerves of the spinal cord from the secondary or tertiary bronchi or from within the esophagus. As further described herein, the one or more electrodes for diaphragm pacing or gastric system stimulation may be in electrical communication with an implantable pulse generator, or may be in electrical communication with a pulse generator positioned external to the patient.

III. Method of Implanting Pulse Generator in Upper Gastrointestinal Tract

In exemplary embodiments, the method of use of the stimulation systems described herein may include at least one electrode implantable within a patient's upper gastrointestinal tract, for example the esophagus or the stomach, and a pulse generator implanted within the esophagus. Various techniques may be performed to implant the electrode or the pulse generator within the upper gastrointestinal tract. For example, techniques similar to those used to perform a bronchoscopy, laryngoscopy, tracheal intubation, or percutaneous catheterization may be performed to position and implant the electrodes or the pulse generator. Similar techniques as described with reference to implantation within the gastrointestinal tract may be performed to implant one or more electrodes or a pulse generator within the airway, such as within the trachea, the primary, secondary, or tertiary bronchus, the bronchioles, or any branch thereof.

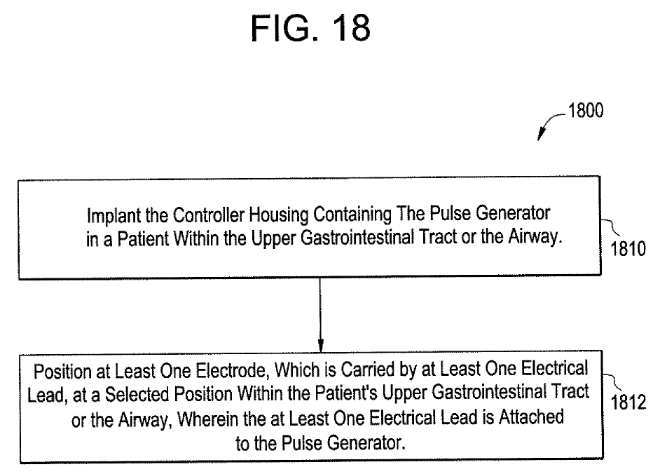

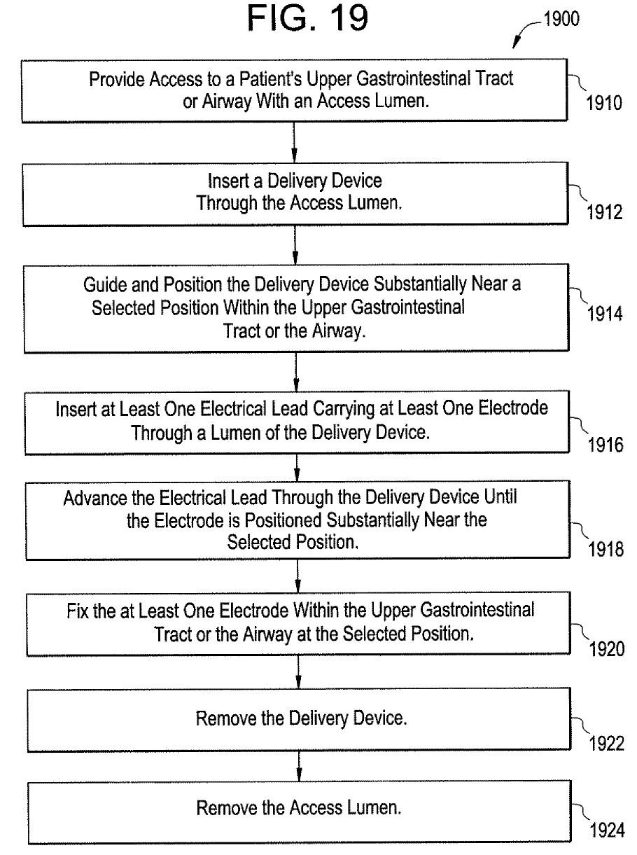

FIG. 18 illustrates a method for implanting a cardiac device according to one embodiment in which the controller housing is implanted within a patient's upper gastrointestinal tract. Flowchart 1800 illustrates an example of a method for implanting a cardiac device including a controller housing and pulse generator and at least one electrode carried by at least one electrical lead, such as those described with reference to FIGS. 2-4.

The method begins at block 1810. At block 1810, the controller housing containing the pulse generator is implanted in the patient within the upper gastrointestinal tract, such as the esophagus. The controller housing may be inserted through the patient's oral or nasal cavity and delivered to the esophagus. The controller housing may be any controller housing operable to perform electrical stimulation or sensing of cardiac, pulmonary, or any other physiologic functions. The controller housing may be implanted using similar procedures as those which may be used to deliver and position an electrode, as described herein. The controller housing may be delivered and positioned using procedures similar to those performed for endoscopies.

The controller housing is anchored within the esophagus to retain the housing at the selected position. The controller housing may be fixed within the esophagus by an anchor device, such as those described herein with reference to FIGS. 8A-8F. The controller housing may further include one or more sensing or stimulation electrodes associated with the casing or the anchor device. Accordingly, anchoring may further serve to improve electrical coupling of any controller housing or anchor electrodes.

Following block 1810 is block 1812, in which at least one electrode carried by at least one lead is positioned at a selected position (also referred to as an “implantation site”) within the upper gastrointestinal tract, such as the esophagus or the stomach. The electrode may be inserted through the patient's oral or nasal cavity, and delivered through into the esophagus to the selected implantation site. The electrode may be any suitable design, such as those described herein with reference to FIGS. 12A-12G.

The order of placement of electrodes within the esophagus for embodiments including more than one electrode may depend, at least in part, on factors such as each electrode's placement relative to one or more other electrodes or the criticality or immediacy of each electrode's purpose. A catheter, endoscope, or other elongated lumen suitable for positioning and delivering medical devices, may be used to deliver the electrical lead and electrode through the esophagus and to the selected implantation site. In one embodiment, an electrode may be delivered by a swallowable capsule. An imaging technique known in the art, such as fluoroscopy, computed tomography, magnetic resonance imaging, x-ray, ultrasound, or position emission tomography may also be utilized to assist with delivering and positioning of the electrode.

Each electrode positioned within the upper gastrointestinal tract may be fixed to retain the electrode at its selected position site and to improve electrical coupling. The electrode or electrodes may be fixed within the airway by an anchor device, such as the embodiments described herein with reference to FIGS. 12A-12G.

Each electrical lead carrying an electrode is attachable to the pulse generator to enable electrical communication therebetween. The electrical lead may be attached prior to implantation, during implantation, or after implantation of the electrodes and/or controller housing (e.g., the pulse generator). Furthermore, in one embodiment, the electrical lead may be permanently integrated within the controller housing, and thus permanently attached. The electrical lead or leads optionally may be fixed within the gastrointestinal tract by a lead securing member, such as embodiments described herein with reference to FIGS. 13A-13D.

The steps described herein need not be performed in the exact order as presented. For example, in some implantation methods, the controller housing may be positioned and anchored prior to the electrodes. In another example, the electrical leads may be attached to the controller housing prior to positioning and anchoring the controller housing, the electrodes, or both the controller housing and the electrodes.

FIG. 19 illustrates a flowchart 1900 describing one method for implanting at least one electrode of an implantable cardiac stimulation system within the upper gastrointestinal tract of a patient, for example the esophagus or the stomach, according to certain embodiments, such as those described herein with reference to FIGS. 2 and 4. However, an electrode may be implanted within a patient's airway, such as within the trachea, the primary, secondary, or tertiary bronchus, the bronchioles, or any branch thereof, in a similar manner.

The method begins at block 1910. At block 1910, access is provided to a patient's esophagus for subsequent insertion of one or more delivery devices and one or more electrical leads each carrying at least one electrode or other sensor. Access may be provided by inserting an access lumen, for example, an endoscope, such as those used when performing esophagogastroduodenoscopies. Moreover, the access lumen may be inserted orally or nasally. This method may be performed while the patient is under general anesthesia, regional anesthesia, or local anesthesia.

Block 1912 follows block 1910, in which a delivery device may be inserted through the access lumen. The delivery device may be any device suitable for aiding with access by a medical device into a lumen of the body, for example, a catheter, guidewire, or combination thereof. In one embodiment, the access lumen, such as an endoscope, may be used to deliver the one or more electrodes without the use of an additional catheter. Exemplary catheters that may be used are torque catheter, steerable catheter, pre-shaped catheter varying by application, deflectable catheter, or catheter and guidewire combination. The delivery device may be a series of catheter systems, by which a first catheter aids in the placement of a second catheter that may carry the electrical lead and electrode, for example. Depending upon the implantation site, example catheter diameters suitable for delivery may range from approximately less than 1 mm to approximately 14 mm. The catheter diameter depends upon its use. For example, a catheter having a diameter of about 1 mm to about 6 mm may be useful for gaining access to and navigating smaller lumens, e.g., for delivering an electrical lead. As another example, a catheter having a diameter of about 2 mm to about 9 mm may be useful for navigating using a endoscope or other imaging device. The diameter of the catheter or other delivery device typically depends upon many factors, including the size of the implantation site, the size of the patient, the configuration of the device being delivered, and the expected duration of the within the lumen.

Following block 1912 is block 1914, in which the delivery device is guided to and positioned substantially near the selected position for implantation. As described herein, the selected implantation site may be at essentially any location within the patient's upper gastrointestinal tract, such as the esophagus or the stomach. In exemplary embodiments, the optimal implantation site for performing electrical stimulation may not be the optimal site for performing sensing. In this situation, a compromise implantation site may be selected, the site correlating to the most important function (e.g., the optimal stimulation site) may be selected, or separate stimulation and sensing electrodes may be implanted. As described herein, the two electrodes may be carried by the same electrical lead or may be carried by individual electrical leads.

One or more imaging techniques may be used to assist guiding the delivery device to the implantation site. Representative examples of suitable imaging techniques include endoscopy, fluoroscopy, computed tomography, magnetic resonance imaging, x-ray, ultrasound, or position emission tomography. The delivery device optionally may include a radiopaque coating or a radiopaque component, as known in the art, to increase visibility and aid in delivery using certain imaging techniques. Other navigation techniques may also be used to aid in delivery. One technique may include the delivery technology developed by superDimension, Ltd. (Herzelia, Israel) known as the in Reach System™, which includes a catheter with a magnetic tracking device calibrated with a computed tomography scan of the patient, allowing for the computed tomography data to assist in guiding the catheter to the implantation site. Another example technique may include the location technique developed by MediGuide, Ltd. (Haifa, Israel) known as the Medical Positioning System™, which includes a catheter or other delivery device having a miniaturized sensor and enables three-dimensional tracking of the device's position. Yet another example guiding technique may include a mapping electrode within the delivery device, such that the mapping electrode may be used to aid in selection of the implantation site. For example, electrical coupling of an electrode, electrical impedance over a wide range of frequencies, and electrical coupling at multiple positions within the gastrointestinal tract may be mapped to identify optimal implantation sites. In one embodiment, one of the electrodes intended to be used for ultimate stimulation and/or sensing may also be used as the mapping electrode, leaving the electrode in place. In another embodiment, an additional mapping electrode may be used with the delivery device and removed prior to positioning and fixing the system electrode or electrodes. For example, a mapping electrode or other sensor may detect one or more intrinsic signals generated by the heart, such as electrical activity or acoustic signals. The mapping electrode may be integrated with the implantable electrical lead or with the delivery device. Additional guiding techniques may also be used, such as measuring the electrical threshold for stimulating the heart or a specific portion thereof. For embodiments that measure the electrical threshold, an algorithm may determine the stimulation gradient, for example by calculating the derivative of the measured threshold along its path.

Block 1916 follows block 1914, in which the electrical lead carrying the one or more electrodes is inserted through the delivery device after the delivery device has been positioned at or near the desired implantation site. As previously described, the delivery device may have a lumen through which the electrical lead may be inserted, such as a catheter. As previously described, the delivery device may be integrated with the electrical lead and electrode, such that delivery and positioning of the delivery device also delivers the electrical lead and electrode. For example, a delivery device may include a first catheter delivered through the esophagus to the implantation site, and a second catheter housing the electrical lead and electrode therein, which is delivered through the first catheter. Accordingly, the delivery device, in some embodiments, may be integrated with the electrical lead and electrode, and all or some of the steps described at blocks 1914-1918 may be performed concurrently.

At block 1918, following block 1916, the electrical lead may be advanced through the delivery device to or substantially near the selected implantation site. As described with reference to insertion/positioning of the delivery device, the method optionally may include imaging techniques or other guiding technologies to assist in delivery of the lead to identify the location of the electrode and its proximity to the selected implantation position.

Block 1920 follows block 1918, in which the electrode may be anchored within the gastrointestinal lumen at the selected implantation position. Any of the described anchor devices may be used to assist anchoring and retaining the electrode at or near the implantation site, such as those described with reference to FIGS. 12A-12G. For example, an electrode embodiment as described with reference to FIGS. 12B and 12C delivered through a catheter or other lumen will have the two or more electrode sub-components compressed within the lumen and the expandable connector under tension during delivery through the delivery device. Upon positioning the electrode at or near the implantation site, the delivery device is removed, which releases the tension on the expandable connector and causes the electrode sub-components to expand radially in contact with the inner walls of the gastrointestinal tract at the selected implantation site. In other electrode embodiments, the anchor devices may require additional action, such as mechanically extending rigid radially extensible members, inflating a balloon or a balloon sleeve, applying heat, radio frequency, electrical, or other energy to change a shape memory alloy-based anchor device, suturing, or stapling, for example. In yet other embodiments, the electrode anchor device may by design anchor without additional action, such as anchor devices configured as hooks, barbs, studs, or adhesive. Optionally, one or more lead securing members may be used to assist retaining the electrical lead within the gastrointestinal tract, such as is described with reference to FIGS. 13A-13D.

Blocks 1922 and 1924 follow block 1920, in which the delivery device and the access lumen are removed upon positioning and fixing the electrode or electrodes. However, in some embodiments, the access lumen and/or the delivery device may be used during implantation of the controller housing (if not implanted prior); thus, the removal steps occurring at blocks 1922 and 1924 may occur subsequent to delivery and implantation of the controller housing.

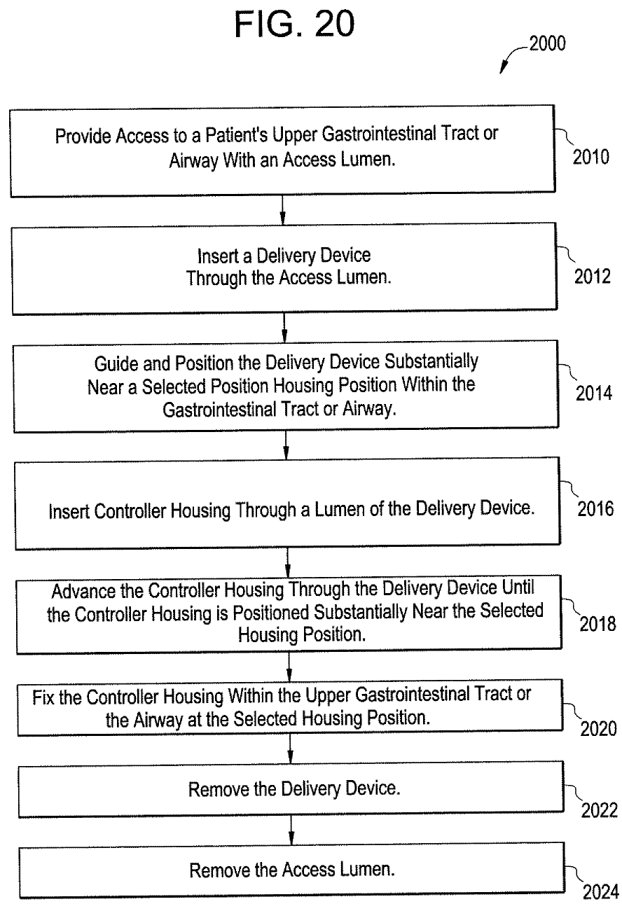

As described herein, the electrical leads may be attached to the controller housing prior to delivery of the electrical leads, or they may be free from the controller housing and attached subsequent to delivery of the electrical leads either before or after delivery of the controller housing. Accordingly, in some embodiments, upon removing the delivery device and the access lumen, the electrical leads may temporarily extend out of the patient's oral or nasal cavity until subsequent attachment to and implantation of the controller housing. Though, in some embodiments, the electrical leads may be retained entirely within the patient's gastrointestinal tract, such as when the controller housing is implanted prior to the electrical leads or otherwise. FIG. 20 illustrates a flowchart 2000 describing one method for implanting a controller housing containing a pulse generator of a cardiac device within the upper gastrointestinal tract of a patient, for example the esophagus or the stomach, such as described herein with reference to FIGS. 2-4 and 6. This method may include steps similar to those described with reference to FIG. 19 for implanting one or more electrodes.

The example method begins at block 2010. At block 2010 access is provided to a patient's esophagus for subsequent insertion of one or more delivery devices and the controller housing. Access may be provided by inserting an access lumen, for example, an endoscope, such as those used when performing esophagogastroduodenoscopies. Moreover, the access lumen may be inserted orally or nasally. This example method may be performed while the patient is under general anesthesia, regional anesthesia, local anesthesia, or performed without anesthesia.

Block 2012 follows block 2010, in which a delivery device may be inserted through the access lumen. The delivery device may be any device suitable for providing access of a medical device into a lumen of the body, such as those described with reference to FIG. 19. In one embodiment of the method, the delivery device may be a series of catheter systems, by which a first catheter aids in the placement of a second catheter that may carry the controller housing, for example. In another embodiment, however, the delivery device may include a guidewire or other supporting device first inserted through the access lumen and a second catheter or other device carrying the controller housing that slides over the guidewire. In yet another embodiment, the delivery device may be a single catheter carrying the controller housing directly to the implantation site without the use of a guidewire or additional catheter.

Following block 2012 is block 2014, in which the delivery device is guided to and positioned substantially near the desired implantation site. The implantation site may be at any point within the patient's upper gastrointestinal tract, such as the esophagus. In one embodiment, in which the pulse generator includes one or more electrodes on the housing or anchor device, the electrode may optionally be used to identify desired implantation site based at least in part on stimulation or sensing functioning as described above. Furthermore, one or more imaging techniques, for example those described with reference to FIG. 19, may optionally be used to assist guiding the delivery device to the implantation site.

Block 2016 follows block 2014, in which the controller housing is inserted through the delivery device after the delivery device has been positioned at or near the desired implantation site. The delivery device may have a lumen through which the controller housing may be inserted, such as a catheter. The controller housing may be integrated with the delivery device at the outset, such that delivery and positioning of the delivery device also delivers the controller housing. Accordingly, for embodiments in which the controller housing is integrated with the delivery device, all or some of the steps described at blocks 2014-2018 may be performed concurrently.

At block 2018, following block 2016, the controller housing may be advanced through, over, or with the delivery device to or substantially near the selected implantation site. As described with reference to delivery of the delivery device, some embodiments may optionally include imaging techniques or other guiding technologies to assist in delivery of the lead to identify the location of the controller housing and its proximity to the selected implantation position.

Block 2020 follows block 2018, in which the controller housing is fixed within the gastrointestinal lumen at the selected implantation position. Any of the anchor devices described herein may be used to assist fixing and retaining the controller housing at or near the implantation site, such as those described with reference to FIGS. 8A-8F or those described with reference to FIG. 19 for implanting an electrode.

Blocks 2022 and 2024 follow block 2020, in which the delivery device and the access lumen are removed upon positioning and anchoring of the controller housing. In some embodiments, however, the access lumen and/or the delivery device may be used during implantation of the electrical lead and electrode (if not implanted prior). Thus, the removal steps occurring at blocks 2022 and 2024 may occur subsequent to delivery and implantation of the electrodes.

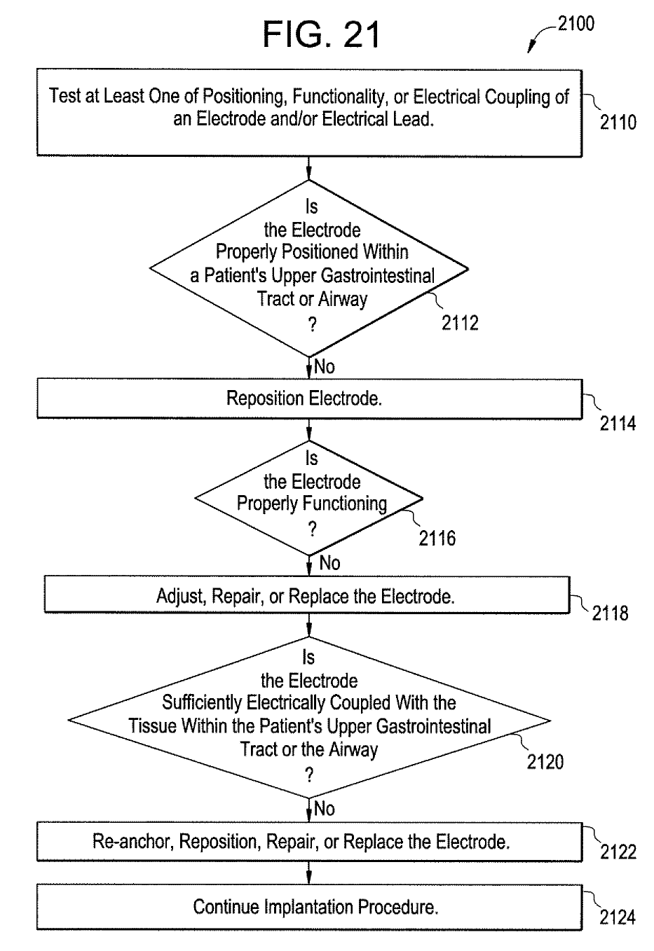

Upon positioning and implantation at least one or more electrodes within the patient's gastrointestinal tract, the functionality, position, and/or electrical coupling of each electrode may be tested. FIG. 21 illustrates a flowchart 2100 describing one method for testing at least one of the positioning, functionality, or electrical coupling of each electrode subsequent to implantation.

The method begins at block 2110. At block 2110, the electrode testing procedures for testing at least one of the positioning of the electrode, the functionality of the electrode, or the electrical coupling of the electrode begin subsequent to implanting the electrode within a patient's upper gastrointestinal tract. This step may include attaching the proximal end of the electrical lead carrying the implanted electrode to external testing electrical circuitry, software, and/or hardware. In other embodiments, the electrical lead may be attached to the controller housing prior to its implantation and the pulse generator may be used at least partially during the testing procedures. While the flowchart 2100 illustrates performing the testing procedures subsequent to implantation of each electrode, the testing procedures may be performed after all electrodes have been implanted, after the controller housing has been implanted, or at any other suitable stage in the implantation methods subsequent to implanting the electrode being tested.

One or more of the decision blocks 2112, 2116, and 2120 follow block 2110, in which at least one of the results of the positioning, functioning, or electrical coupling testing is queried. Each of the steps described at blocks 2112, 2116, or 2120 are not required to be performed and certain methods used may perform only a subset of the testing and determination procedures.

At decision block 2112, it is determined whether the electrode is properly positioned. This determination may be performed using any of the imaging techniques, guiding techniques, or electrical signal monitoring described herein. If it is determined that the electrode is not positioned properly, then block 2114 follows, in which the electrode may be repositioned according to any of the electrode placement methods described herein, such as those described with reference to FIG. 19. Alternatively, if it is determined that the electrode is positioned properly, then block 2116 follows.

At decision block 2116, it is determined whether the electrode is properly functioning. This determination may be performed using externally located testing circuitry, electronic controllers, software, hardware, or the like, as is suitable for performing electrode testing. Electrode functions, such as conductivity, electrical stimulation functioning, or sensing functioning, may be tested by this procedure. For example, whether the electrode stimulation is within a pre-defined acceptable range, or whether the electrode stimulation threshold is stable. Further, safe operation may be tested at this stage as well. If it is determined that the electrode is not functioning properly, then block 2118 follows, in which the electrode may be adjusted, repaired, or replaced. Alternatively, if it is determined that the electrode is functioning properly, then block 2120 follows.

At decision block 2120, it is determined whether the electrode is sufficiently electrically coupled with the tissue at the selected implantation site. Similar to testing the functionality, external to hardware, software, and/or the pulse generator may be used to perform the electrical coupling testing. In one example, the electrical impedance is measured between the implanted electrode and another electrode operating as a reference electrode, and using electronic circuitry, such as a resistance-capacitance-inductance meter, as known in the art. If it is determined that the electrode is not properly coupled, then block 2122 follows, in which the electrode may be re-anchored, repositioned, repaired, or replaced. Alternatively, if it is determined that the electrode is coupling properly, then block 2124 follows.

At block 2124 the testing procedures are completed and subsequent implantation steps may be performed as necessary, such as implanting additional electrodes, the controller housing, or attaching the electrical leads to the housing, as is described herein with reference to FIGS. 18-20, for example.

As illustrated by FIG. 21, a testing method may be performed subsequent to implantation of each electrode. The method may be performed prior to attaching the electrical leads to the controller housing, and be tested using external testing circuitry and hardware. In another example, the method may be performed subsequent to attaching the electrical leads to the controller housing, either prior to or subsequent to implanting the controller, and may at least partially use the pulse generator to perform the testing. In other testing methods, the testing may be performed only after all of the electrodes are implanted.

Although FIGS. 18-21 are described with reference to implanting a controller housing or electrodes within a patient's upper gastrointestinal tract, the same or substantially similar steps may be performed to implant a controller housing or electrodes within a patient's airway, such as within the trachea, the primary, secondary, or tertiary bronchi, the bronchioles, or a combination thereof such as is illustrated in FIGS. 2-6.

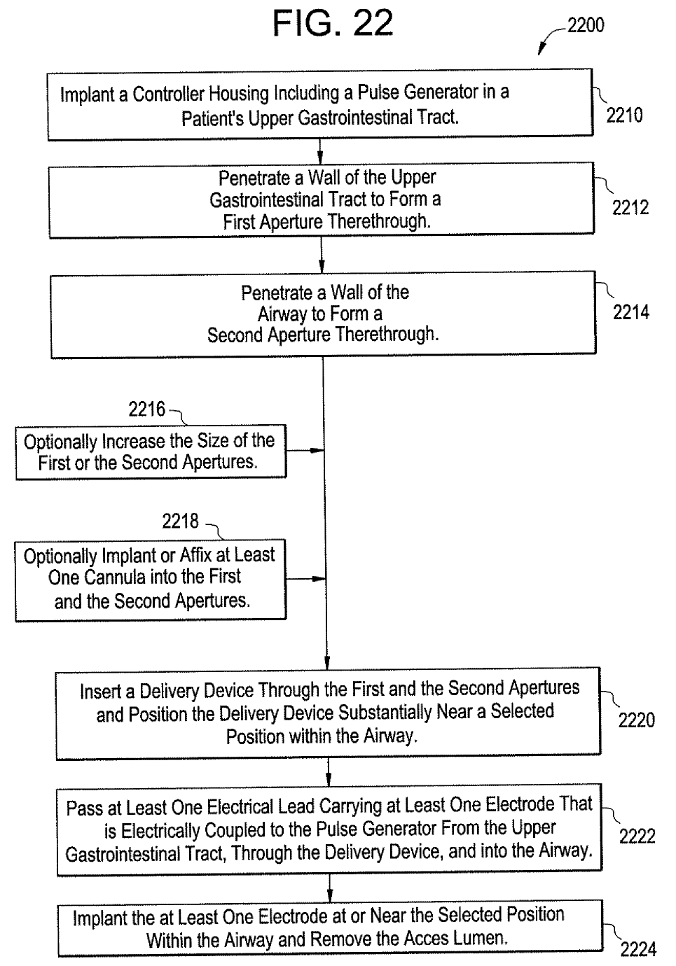

FIG. 22 illustrates a flowchart 2200 describing one method for implanting a controller housing containing a pulse generator of a cardiac device within the upper gastrointestinal tract of a patient and passing at least one electrical lead carrying at least one electrode to the patient's airway, such as described herein with reference to FIGS. 7B-7C.

The exemplary method begins at block 2210. At block 2210 a controller housing including a pulse generator is implanted within a patient's upper gastrointestinal tract, such as within the esophagus. The controller housing may be implanted by methods similar to that described with reference to FIG. 20.

Following block 2210 is block 2212, in which the wall of the gastrointestinal tract is penetrated, forming a first aperture therein. The first aperture may be formed in the esophagus wall using a needle or wire, or a cutting device, such as a blade or scissors, for example. Following block 2212 is block 2214, in which a second aperture is formed in the trachea wall, between the cartilage rings of the trachea. The first and second apertures may be formed independently, or may be formed in a single step, penetrating both the esophagus and the trachea at the same time. The order in which the esophagus and the trachea are penetrated at blocks 2212 and 2214, respectively, may be reversed, such as when penetrating the trachea from within the trachea rather than the esophagus. Moreover, in embodiments including multiple electrical leads passing between the esophagus and the airway, multiple apertures may be formed through the esophagus and the trachea, an electrical lead passing through each.

Block 2216 follows block 2214, in which the diameter of the apertures may optionally be increased using a fenestrator, catheter tip, blade, scissors, needle, or wire, or other suitable device for forming and/or opening an aperture in a human lumen.

At block 2218, following block 2216, at least one cannula may optionally be implanted in the first and second apertures, or affixed to the inner walls of the esophagus and trachea, such as is described with reference to FIGS. 7C, 14-16. Though, in other embodiments, the one or more electrical leads may pass directly through the apertures, without implanting a cannula.

At block 2220, following block 2218, a delivery device may be guided through the first and second apertures and positioned substantially near the selected electrode implantation position within the patient's airway. In one embodiment, the delivery device is inserted orally or nasally into the esophagus, guided from the esophagus through the apertures (and optionally the cannula), and into the airway to the implantation site. The delivery device may be guided and positioned substantially near the selected implantation site using methods similar to those described with reference to FIG. 19 describing electrode implantation. As described with reference to FIG. 19, the delivery device may optionally be positioned using imaging techniques or other guiding technologies. In another embodiment, however, the delivery device may be inserted orally or nasally into the trachea and through the apertures from the trachea.

Block 2222 and 2224 follow block 2220, in which the electrical lead carrying the one or more electrodes is inserted through the delivery device, delivered to the implantation site, and the electrode is fixed therein. Upon positioning the delivery device at the implantation site, the electrical lead and electrodes may be fixed in the same manner as described with reference to FIG. 19. Upon implantation of the one or more electrodes, the delivery device may be removed, pulled through the esophagus or through the trachea, depending upon initial insertion method. As stated, in another embodiment, the electrode may be first positioned and fixed at the selected implantation site within the airway, and then passed through the apertures to the esophagus, by the delivery device, for connecting with the pulse generator.

IV. Implantable Electrodes and Electrical Leads Attachable to a Pulse Generator Implantable Subcutaneously

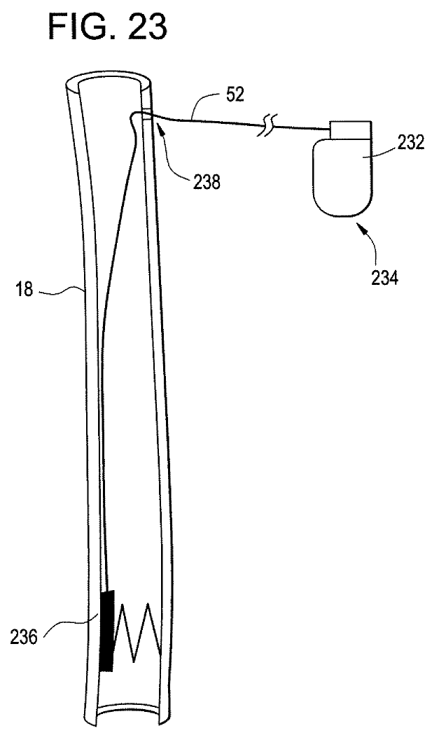

In another embodiment, a controller housing including a pulse generator may be implantable at a subcutaneous location within the patient, and at least one electrical lead carrying at least one electrode fixable within the upper gastrointestinal tract or the airway, may pass through, or communicate wirelessly at, an area of the patient's esophagus, trachea, or a primary bronchus. FIG. 23 illustrates one embodiment of an implantable cardiac stimulation system having a controller housing 232 including a pulse generator 234 implantable subcutaneously and at least one electrode 236 implantable within the esophagus 18. Thus, this embodiment minimizes the components implanted within the gastrointestinal tract, but does require an invasive surgical procedure for implantation of the controller housing 232. For example, the controller housing 232 may be surgically implanted approximately in a patient's pectoral region and a subcutaneous tunnel formed from the controller housing 232 to the patient's esophagus 18. In other variations of this embodiment, another subcutaneous location may be selected as the implantation site for the controller housing.

The pulse generator 234 of this embodiment may be operable to perform some or all of the same functions described herein, such as those described with reference to FIGS. 2-4 describing a pulse generator implanted within an esophagus. For example, the pulse generator 234 may perform electrical stimulation through the one or more attachable electrical leads and electrodes 236, such as is used to perform atrial cardiac pacing, ventricular cardiac pacing, dual chamber cardiac pacing, cardiac resynchronization therapy, cardioversion, and/or defibrillation. The pulse generator 234 may be operable to sense or measure cardiac electrical activity, other cardiac activity, and/or other physiological parameters.

As used with this embodiment the pulse generator 234 may be a conventional implantable pulse generator suitable for subcutaneous implantation, as is commercially available; which may also commonly be referred to as an “implantable pulse generator” or an “implantable cardioverter-defibrillator.” However, the electrical circuitry, software, and hardware of the pulse generator 234 may be altered or adapted for operation with electrodes implantable within the upper gastrointestinal tract or airway, as compared to conventional implantable pulse generators used with electrodes in direct contact with the heart. The controller housing 232 may be proportioned to have a substantially flat shape to ease placement subcutaneously and avoid discomfort to the patient. The controller housing 232 may be hermetically sealed, electrically isolated, biocompatible, in order to operate safely and to withstand the biological environment within which it may be implanted.

The pulse generator 234 is electrically coupled to at least one electrical lead 52, carrying at least one electrode 236 positionable and fixable at a selected position within the patient's upper gastrointestinal tract, such as the esophagus. The electrode or electrodes 236 may be positioned at or near the distal end of the electrical leads 52, as illustrated in FIG. 23. However, in other embodiments, an electrode may be positioned at another point distanced from the lead's distal end. In another embodiment, the pulse generator 234 may be operated in combination with one or more gastrointestinally implanted electrodes 236 and with one or more conventionally implanted electrodes, such as transvenous electrodes, epicardial electrodes, or epidermally placeable electrodes. Moreover, in some embodiments, one or more electrodes may additionally, or alternatively, be implantable within the patient's airway. For example, an electrical lead may pass from within the esophagus to the trachea, as described herein, or may pass directly from the controller housing 232 to the trachea through a subcutaneous tunnel in a similar manner.

A subcutaneous tunnel, through which the one or more electrical leads 52 may pass, may be surgically formed between the controller housing 232 implantation site, for example near the pectoral region, and a junction 238 at the in the esophagus 18. As illustrated, the electrical lead or leads 52 may pass through one or more apertures formed in the esophagus 18 at the junction 238 and into one or more locations within the gastrointestinal tract. The aperture may be formed in any manner similar to those described with reference to FIGS. 7B-7C and 22, and may optionally include one or more cannulae similar to that described with reference to FIGS. 7C, 14-16, and 22. Alternatively, rather than passing through an aperture, the electrical lead 52 may communicate wirelessly across the junction 238, similar to that described with reference to FIG. 17. In embodiments including multiple electrodes, a single lead may be coupled to the pulse generator 234 and split into multiple leads carrying each one or more electrodes to respective selected implantation positions within the gastrointestinal tract. In other embodiments, however, each electrode may be carried by individual leads, which may be optionally bundled to ease the passage through an aperture, or to simplify wireless communication, at the junction 238. The electrical leads 52 used in these embodiments may be substantially similar to other electrical leads described herein. The embodiment illustrated in FIG. 23 is provided for exemplary purposes; other electrode and controller housing positioning and configurations are envisioned. For example, the electrodes may be positioned at any selected electrode position, such as shown in FIGS. 2-6.

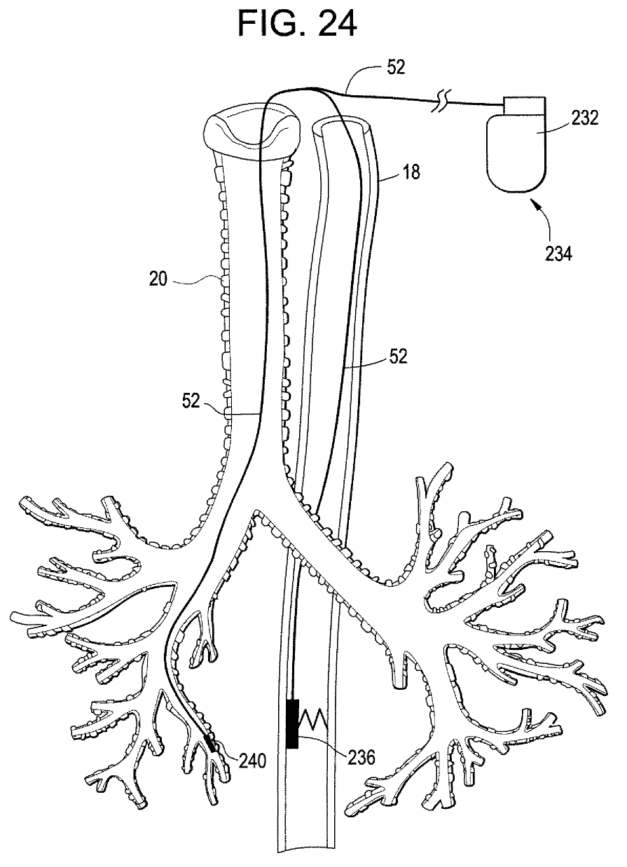

FIG. 24 illustrates another embodiment of an implantable stimulation system having a controller housing 232 including a pulse generator 234 implantable subcutaneously and at least one electrode 236 implantable within the upper gastrointestinal tract and at least one electrode 240 implantable within the airway. According to this embodiment, an aperture may be formed through a wall of the upper gastrointestinal tract, such as in the esophagus 18, for the delivery of the electrode 236 implantable within the upper gastrointestinal tract. An aperture may also be formed in a wall of the airway, such as in the trachea 18 or the left or right primary bronchus 30, 32, for the delivery of the electrode 240 implantable within the airway. The aperture may be formed in a manner similar to those described with reference to FIGS. 7B-7C and 22, and may optionally include one or more cannulae similar to that described with reference to FIGS. 7C, 14-16, and 22. In one embodiment, at least two electrical leads 52 may connect to the controller housing 232 implanted subcutaneously, one carrying the gastrointestinal implanted electrode 236 and the other carrying the airway implanted electrode 240. However, in other embodiments, a single electrical lead 52 may be connected to the controller housing 232 which splits into at least two sub-leads for carrying the gastrointestinal implanted electrode 236 and the airway implanted electrode 240. A cannula optionally may be implanted in each of the apertures formed in the wall of the gastrointestinal tract and the wall of the airway, in a manner similar to that described with reference to FIG. 23. In another embodiment, a tissue interface operable for wirelessly transmitting signals across one or both of the gastrointestinal wall or the airway wall may be employed rather than forming apertures for passing electrical leads therethrough.

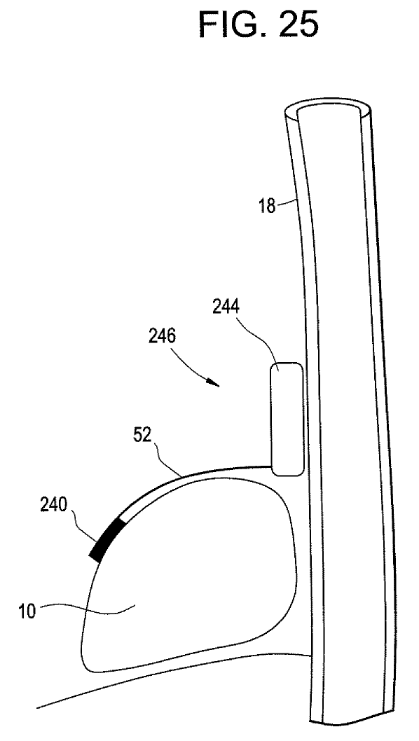

FIG. 25 illustrates another embodiment of a stimulation system that may be implantable subcutaneously. Though, as illustrated in FIG. 25, a controller housing 244 including a pulse generator 246 may be secured to an exterior wall of the upper gastrointestinal tract, such as an exterior esophagus 18 wall, or in another embodiment to the exterior wall of the airway, such as the trachea. The controller housing 244 may be retained by suture, staples, barbs, hooks, studs, adhesive, or any combination thereof, for example. At least one electrical lead 52 electrically coupled to the pulse generator 246 may carry at least one electrode 240 that is positionable and fixable at a position at a position on the epicardium of a patient's heart 10, in a manner similar to the embodiment described with reference to FIG. 6, for example. The controller housing 244 may be delivered to an exterior wall of the esophagus and the at least one electrical lead 52 and electrode 240 may be delivered to the heart 10 by delivery orally or anally into the upper gastrointestinal tract, through the esophagus 18, and through an aperture formed in the esophagus 18 using a delivery device, such as a catheter or an endoscope, for example. This embodiment may further be used with one or more airway implanted electrodes, gastrointestinal implanted electrodes, or conventionally implanted electrodes, such as transvenous electrodes or epidermally or subcutaneously placeable electrodes.

Cannula

In one embodiment, the upper gastrointestinal tract, such as the esophagus, may be penetrated and one or more apertures may be formed therethrough for passing at least one electrical lead carrying at least one electrode from the subcutaneously implanted controller housing and into the upper gastrointestinal tract. A cannula similar to those described with reference to FIGS. 14-16, but dimensioned for implantation only through a wall at any point of the upper gastrointestinal tract, may be used according to these embodiments. Cannula designs and methods other than those described herein may be employed to aid in the retention of electrical leads and sealing the thoracic cavity from the gastrointestinal tract. For example, certain embodiments may not include a cannula, but may allow the one or more electrical leads to pass directly through the aperture formed in the wall of the upper gastrointestinal tract. Furthermore, in other embodiments, other means for sealing the aperture may be used, such as an adhesive, a membrane, suturing, or stapling, for example.

In some embodiments in which one or more devices are passed from within the upper gastrointestinal tract to a subcutaneous position within the body, contamination from within the tract may be prevented and/or treated to promote a more sterile environment. For example, in some embodiments, the electrode, electrical lead, cannula, or other device may be covered with a sterile sleeve prior to subcutaneous insertion from the esophagus. In other embodiments, the electrode, electrical lead, cannula, or other device may be treated (e.g., coated) with an antimicrobial material, such as antiseptic and/or antibiotic agent. Furthermore, the patient may be treated with antibiotics, steroids, or other pharmaceutical agents systemically or by inhalation, prior to and/or after the implantation procedure. Devices, such as electrodes, leads, or a controller housing implantable within the upper gastrointestinal tract may be similarly coated or treated to prevent infection and scarring within the airway.

Wireless Tissue Interface

One embodiment may include a tissue interface adaptable for wirelessly communicating between the subcutaneously implanted pulse generator and the electrodes implanted within the upper gastrointestinal tract or the airway, rather than forming an aperture therethrough. A tissue interface similar to that described with reference to FIG. 17 may be used for wirelessly communicating according to this embodiment. Accordingly, an implantable device may include a subcutaneous electrical lead portion from the pulse generator 234 to the wireless tissue interface and a gastrointestinal lead portion (or an airway lead portion) carrying at least one electrode 236 from the tissue interface within the upper gastrointestinal tract. Moreover, as described above, one or more of the electrical lead portions may not be necessary in embodiments which communicate wirelessly directly from the pulse generator to the electrode or directly from the tissue interface to the electrode. The wireless tissue interface described as being operable for communications across the esophagus wall also may be used for communications across the airway wall for embodiments including electrodes implantable within the airway.

V. Method of Implanting a Pulse Generator Subcutaneously

In one aspect, the system may include at least one electrode implantable within a patient's upper gastrointestinal tract, for example the esophagus, or airway, for example the primary, secondary, or tertiary bronchus, or the bronchioles, and a controller housing containing a pulse generator implantable subcutaneously and external to the patient's upper gastrointestinal tract and airway. Various techniques may be performed to implant an electrode within the upper gastrointestinal tract and airway or to implant the controller housing subcutaneously. For example, techniques similar to those described herein with reference to FIGS. 11 and 12 may be performed to position and implant the one or more electrodes. Additional methods are described for implanting a controller housing subcutaneously.

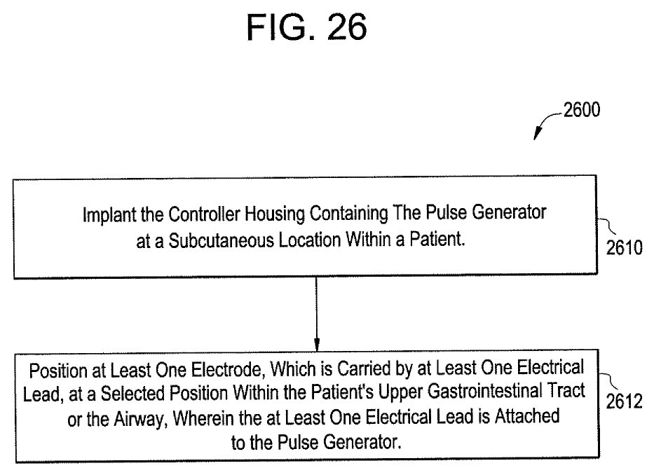

FIG. 26 illustrates a flowchart 2600 describing one example of a method for implanting an implantable cardiac stimulation system including a controller housing and at least one electrode carried by at least one lead, such as the embodiments described with reference to FIGS. 23-24.

The method begins at block 2610. At block 2610 the controller housing is implanted subcutaneously. An incision may be made and the controller housing may be implanted in a manner similar to methods used for commercially available implantable controller housings, as are known. The controller housing may be any example controller housing operable to perform electrical stimulation or sensing of cardiac, pulmonary, or any other physiologic functions, such as the embodiment described with reference to FIG. 23.

Block 2612 follows block 2610, in which at least one electrode carried by an electrical lead is positioned at an implantation site within the upper gastrointestinal tract or the airway. Although an implantation site within the esophagus is described, an electrode may be implanted at any other position within the upper gastrointestinal tract or the airway. The electrical lead may be delivered from the controller housing through an aperture formed in the esophagus. Alternatively, the electrical lead may be inserted through the patient's oral or nasal cavity, and delivered through the esophagus to the selected implantation position in the upper gastrointestinal tract, such as is described with reference to FIG. 19. The electrode may be an electrode embodiment described herein, such as those described with reference to FIGS. 12A-12G. As described herein, some embodiments may include more than one electrode; thus, each electrode is positioned at its implantation site within the esophagus at block 2612 of this example method. The order of placement of electrodes within the esophagus for embodiments including more than one electrode may be at least partially dependent upon factors such as each electrode's placement relative to other electrodes or the criticality or immediacy of each electrode's purpose. A delivery device, such as a catheter, endoscope, or other elongated lumen suitable for positioning and delivering medical devices, may be used to deliver the electrode and lead through the esophagus and to the implantation site. The delivery device may be inserted into the esophagus through the aperture formed in the esophagus wall or may be inserted orally or nasally into the esophagus. In one embodiment, an imaging technique known in the art, such as fluoroscopy, computed tomography, magnetic resonance imaging, x-ray, ultrasound, position emission tomography, for example, may also be performed to assist in the delivery and positioning of the electrode.

Each electrode positioned within the esophagus may be fixed within the esophagus to retain the electrode at the desired implantation site and to improve electrical coupling. The electrode or electrodes may be fixed in any manner described herein, such as with reference to FIG. 19.

Each electrical lead carrying an electrode may be coupled to the pulse generator to enable electrical communication therebetween. Accordingly, in one embodiment, an electrical lead delivered by way of a delivery device passing through the catheter may already pass through a subcutaneous tunnel created from the controller housing to the aperture in the esophagus, and may simply be attached to the controller housing if not already coupled. In another embodiment, however, the electrical lead may have been inserted orally or nasally into the esophagus. For this embodiment, the lead may be snared or otherwise pulled through the aperture formed in the esophagus wall, through the subcutaneous tunnel, and attached to the subcutaneously implanted controller housing. This step is optional, and may not be required for certain embodiments. For example, in some embodiments, the electrical lead or leads may be permanently affixed to the controller housing. In other embodiments, wireless communication is used instead of electrical leads.

These steps need not be performed in the exact order as presented. For example, in some implantation methods, the electrodes may be positioned and anchored prior to implanting the controller housing. In another example, the electrical leads may be attached to the controller housing prior to implanting the controller housing, the electrodes, or both.

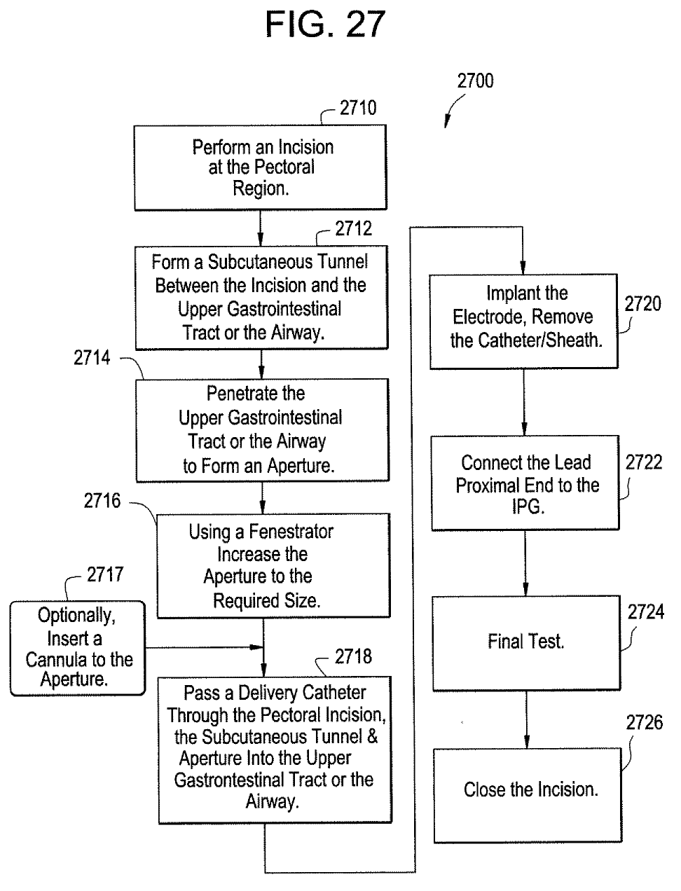

FIG. 27 illustrates a flowchart 2700 describing one method for implanting a controller housing of a cardiac device subcutaneously, for example at or near the pectoral region, according to another embodiment, such as described with reference to FIGS. 22-23.

The method begins at block 2710. At block 2710 an incision is made through the patient's epidermis and dermis for implanting the controller housing at the controller housing implantation site. In one embodiment, the incision may be made at or near the patient's pectoral region. Alternatively, the incision may be made at another area suitable for access to the implantation site.

Block 2712 follows block 2710, in which a subcutaneous tunnel may be formed between the controller housing implantation site and a point on the upper gastrointestinal tract, such as the esophagus, using a tunneling device and procedure as known in the art. Although this example describes implanting an electrode within the esophagus, one or more electrodes may be placeable at other points within the upper gastrointestinal tract or within the airway. The subcutaneous tunnel allows one or more electrical leads to pass subcutaneously from the controller housing to the esophagus. Accordingly, the subcutaneous tunnel may have a diameter large enough at least for the electrical lead or leads to exist therein, and optionally large enough for an electrode delivery device, such as a catheter, to pass therethrough. The size of the subcutaneous tunnel may be adjusted by adjusting the size of the tunneling device or by subsequent enlarging procedures using the tunneling device, for example.

At block 2714, following block 2712, the esophagus may be penetrated and an aperture formed therein for passing the one or more electrical leads therethrough and into the patient's upper gastrointestinal tract. A point of penetration may be determined using one or more imaging and/or guiding technologies, as described herein, or by palpation. The point of penetration may be accessed and the aperture formed from the subcutaneous tunnel in one embodiment. In another embodiment, the penetration may be made from within the esophagus and into the subcutaneous tunnel. The penetration may be made and the aperture formed using a needle, wire, spike, blade, forceps, or the like, which may optionally be inserted through a delivery device, such as a catheter, to the point of penetration.

Following block 2714 is block 2716 in which the size of the aperture may be adjusted, based on the intended electrode configuration for the device. The passage diameter may be increased using a fenestrator, catheter tip, forceps, blade, tunneling device, or other suitable device for forming or opening an aperture in a human lumen. As described with reference to FIGS. 13-15, a cannula optionally may be implanted in the aperture, or affixed to the exterior and/or inner wall, of the esophagus at block 2717.

At block 2718, following block 2716, a delivery device may be guided to and positioned substantially near the selected electrode implantation position within the patient's esophagus. In one embodiment, the deliver device is guided from the subcutaneous tunnel, through the aperture (and optionally the cannula), and into the esophagus to the implantation site. In another embodiment, however, the delivery device may be inserted orally or nasally. The delivery device may be guided and positioned substantially near the selected implantation site using methods similar to those described with reference to FIG. 19 describing electrode implantation. The delivery device optionally may be positioned using imaging techniques or other guiding technologies.

Block 2720 follows block 2718, in which the electrical lead carrying the one or more electrodes is inserted through the delivery device, delivered to the implantation site, and the electrode is fixed therein. Upon positioning the delivery device at the implantation site, the electrical lead and electrodes may be fixed in the same manner as described with reference to FIG. 19. Upon implantation of the one or more electrodes, the delivery device may be removed, pulled through the esophagus aperture and subcutaneous tunnel or through the oral or nasal cavity, depending upon initial insertion method.

Block 2722 follows block 2720, in which each electrical lead carrying an electrode is coupled to the pulse generator to enable electrical communication therebetween. The electrical leads may be coupled to the controller housing in the same manner described with reference to FIG. 26. This step is optional and may not be required for certain embodiments. For example, in some embodiments, the electrical lead or leads may be permanently affixed to the controller housing. In other embodiments, wireless communication may be used instead of electrical leads.

At block 2724, following block 2722, the electrode testing procedures for testing at least one of the positioning of the electrode, the functionality of the electrode, or the electrical coupling of the electrode may be performed as described with reference to FIG. 21. If the embodiment includes an aperture formed in the esophagus and a cannula implanted therein, the positioning, stability, and seal of the cannula may be optionally tested at this step. Following block 2724, after the testing procedures are performed, the incision may be closed, and the implantation method is completed at block 2726.

These steps need not be performed in the exact order as presented. For example, in some implantation methods, the electrodes may be positioned and anchored prior to implanting the controller housing. In another method, the electrical leads may be attached to the controller housing prior to implanting the controller housing, the electrodes, or both. In yet another method, the testing procedures may be performed after implanting each electrode or after implanting a cannula, for example.

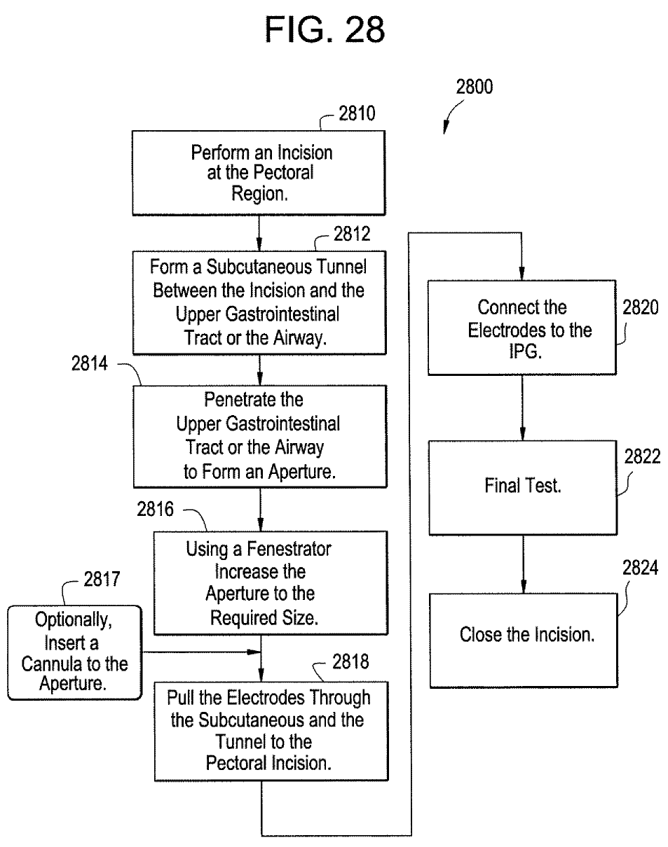

FIG. 28 illustrates a flowchart 2800 describing another suitable method for implanting a controller housing subcutaneously, for example, at or near the pectoral region, and pulling one or more electrical leads from within the esophagus, according to various embodiments, such as those described with reference to FIGS. 23-24.

The method may begin at block 2810. The steps performed at blocks 2810-2817 may be performed in a substantially similar manner as the steps described with reference to blocks 2710-2717 of FIG. 27. However, for the implantation method described with reference to FIG. 28, the electrical leads may be implanted through an oral or nasal cavity, in a substantially similar manner as is described with reference to FIG. 19. Upon implantation, the electrical leads may remain within the esophagus.

Block 2818 follows block 2817, in which a retrieval lumen, such as a catheter, and/or a retrieval tool may grasp the proximal end of the electrical lead within the esophagus and pull the lead through the aperture to attach to the subcutaneously implanted controller housing, such as is described with reference to FIGS. 16A-16B. An endoscope, such as is used for an esophagogastroduodenoscopy, or other visualization, imaging, or guiding techniques, may aid in grasping and retrieving the electrical lead by the retrieval tool. As described with reference to FIG. 27, in one embodiment, the esophagus may be penetrated from within the esophagus to form the aperture, rather than from within the subcutaneous tunnel. In another embodiment, the proximal end of the electrode or electrodes may remain external to the patient, for example passing out of the patient's oral or nasal cavity. The retrieval tool may be passed through the aperture from the subcutaneous tunnel and out of the same orifice, allowing the grasping or temporary coupling of the electrical lead to be performed externally. Upon grasping, the retrieval tool may be pulled through the aperture and the subcutaneous tunnel, for attachment of the electrical lead with the controller housing. In another embodiment, the retrieval tool initially may be inserted through the patient's esophagus, such as through the oral or nasal cavity, and then through the aperture into the subcutaneous tunnel for delivering the and attaching the electrical lead to the controller housing. In one embodiment including a cannula, such as the cannula described with reference to FIGS. 16A-16B, the cannula may be affixed to the inner or exterior wall of the esophagus when the electrical lead is pulled, as described more completely with reference to FIGS. 16A-16B.

The steps performed at blocks 2820-2824 may be performed in a substantially similar manner as the steps described with reference to blocks 2722-2726 of FIG. 27.

VI. Method of Electrically Stimulating a Heart

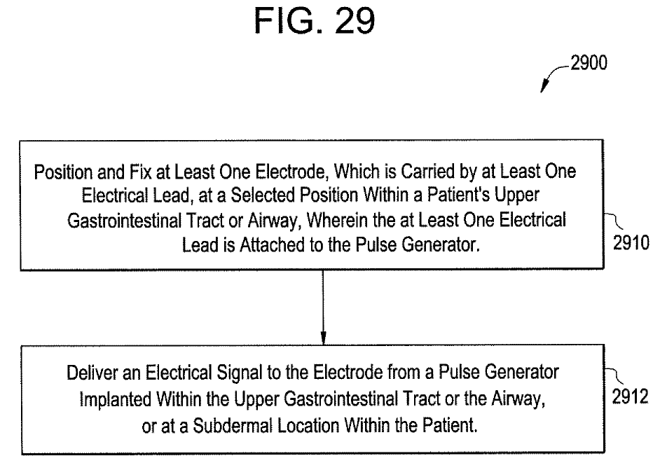

FIG. 29 illustrates a flowchart 2900 describing one method for stimulating a patient's heart using example implantable cardiac stimulation system as described herein, such as those described with reference to FIGS. 2-6 and 23-24.

The method begins at block 2910. At block 2910, at least one electrode is positioned and fixed at a selected position within the patient's upper gastrointestinal tract, such as the esophagus, or the airway, such as the trachea, primary, secondary, or tertiary bronchus, bronchioles, or any branch thereof. The one or more electrodes may be carried by one or more electrical leads, respectively, which are attached to a controller housing including a pulse generator implanted within the patient. The electrode and electrical lead may be positioned and fixed by example methods and devices described herein, such as those described with reference to FIGS. 12A-12G and 13A-13D.

Following block 2910 is block 2912, in which an electrical stimulation signal from a pulse generator is delivered from the pulse generator. The pulse generator may be housed within the control housing and implantable within the patient's upper gastrointestinal tract, such as the esophagus, or airway, such as the trachea or primary bronchus, or subcutaneously external to the patient's upper gastrointestinal tract and airway, such as within the pectoral region or affixed to a wall of the gastrointestinal tract or airway. The electrical stimulation signal may be effective for performing cardiac pacing, cardiac defibrillation, anti-tachycardia pacing, cardioversion, cardiac resynchronization therapy, or any combination thereof. In other embodiments, other tissues, such as the phrenic nerve, diaphragm, or upper gastric system may be stimulated for treating respiratory paralysis, for performing gastric electric stimulation, or to perform diaphragm pacing.

Publications cited herein are incorporated by reference. Modifications and variations of the methods and devices described herein will be obvious to those skilled in the art from the foregoing detailed description. Such modifications and variations are intended to come within the scope of the appended claims.

I claim:

1. An implantable cardiac stimulation system comprising:
a controller housing comprising a pulse generator, said controller housing being adaptable for subcutaneous implantation;
at least one electrical lead attachable to said pulse generator; and
at least one electrode, which is carried by said at least one electrical lead, said at least one electrode positionable and fixable at a first selected position within a patient's upper gastrointestinal tract.

2. The system of claim 1, further comprising a cannula adaptable for passage of said at least one electrical lead through a wall of the esophagus of said upper gastrointestinal tract.

3. The system of claim 2, wherein said cannula is implantable in said wall of said esophagus and adaptable to substantially exclude passage of air or biological fluids through an aperture in said wall formed during implantation of said cannula.

4. The system of claim 1, further comprising a tissue interface for wirelessly communicating an electrical signal through a wall of said upper gastrointestinal tract.

5. The system of claim 4, wherein said at least one electrical lead comprises a subcutaneous lead portion attachable to said pulse generator and adaptable to electrically communicate with said tissue interface external to said upper gastrointestinal tract, and a gastrointestinal lead portion carrying said at least one electrode and adaptable to electrically communicate with said tissue interface within said upper gastrointestinal tract.

6. The system of claim 1, further comprising a second electrode which is carried by a second electrical lead, said second electrode being positionable and fixable at a second selected position within said upper gastrointestinal tract.

7. The system of claim 1, further comprising a second electrode which is carried by a second electrical lead, said second electrode being positionable and fixable at a position within the trachea, the bronchus, the bronchioles, or a branch thereof of the patient's airway.

8. The system of claim 7, further comprising a cannula adaptable for passage of said second electrical lead through a wall of said trachea or said bronchus.

9. The system of claim 8, wherein said cannula is implantable in said wall of said trachea or said bronchus and adaptable to substantially exclude passage of air or biological fluids through an aperture in said wall formed during implantation of said cannula.

10. The system of claim 7, further comprising a tissue interface for wirelessly communicating an electrical signal through a wall of said trachea or said bronchus.

11. The system of claim 1, wherein said pulse generator is operable to deliver one or more electrical pulses effective in cardiac pacing, cardiac defibrillation, cardioversion, cardiac resynchronization therapy, or a combination thereof.

12. The system of claim 1, further comprising an anchor for securing said at least one electrode within said upper gastrointestinal tract.

13. The system of claim 1, wherein said at least one electrode is further operable for sensing electrical cardiac activity, cardiac movement, electrode position, temperature, pH, pressure, ultrasonic measurements, or a combination thereof.

14. An implantable cardiac stimulation system comprising:
a controller housing comprising a pulse generator, said controller housing being adaptable for implantation at a selected housing position within a patient's upper gastrointestinal tract, said controller housing being proportioned to substantially permit fluid and solid flow through said upper gastrointestinal tract about said selected housing position;
at least one electrical lead attachable to said pulse generator; and
at least one electrode carried by said at least one electrical lead.

15. The system of claim 14, wherein said at least one electrode is positionable and fixable at a first selected electrode position within said upper gastrointestinal tract.

16. The system of claim 14, further comprising at least one anchor for fixing said controller housing at said selected housing position within said upper gastrointestinal tract.

17. The system of claim 14, wherein said controller housing comprises at least two substantially rigid sub-cases and at least one flexible connector between each rigid sub-case.

18. The system of claim 14, wherein said controller housing comprises an at least partially flexible casing.

19. The system of claim 14, wherein said controller housing comprises a reattachably detachable portion.

20. The system of claim 19, wherein said reattachably detachable portion comprises a power source, a memory, a processor, electrical circuitry, or a combination thereof.

21. The system of claim 14, wherein said pulse generator is operable to deliver one or more electrical pulses effective in cardiac pacing, cardiac defibrillation, cardiac resynchronization therapy, or a combination thereof.

22. The system of claim 14, wherein said controller housing further comprises at least one electrode.

23. The system of claim 14, further comprising at least a second electrode, which is carried by at least a second electrical lead.

24. The system of claim 23, wherein said second electrode is positionable and fixable at a second selected electrode position within said upper gastrointestinal tract.

25. The system of claim 23, wherein said second electrode is positionable and fixable at a position within the trachea, the bronchus, the bronchioles, or a branch thereof, of the patient's airway.

26. The system of claim 25, further comprising at least one cannula adaptable for passage of said second electrical lead through a wall of said upper gastrointestinal tract or through a wall of said trachea or said bronchus, or a combination thereof.

27. The system of claim 25, further comprising at least one tissue interface for wirelessly communicating an electrical signal through a wall of said upper gastrointestinal tract, through a wall of said trachea or said bronchus, or a combination thereof.

28. The system of claim 25, wherein said second electrode is passable from said upper gastrointestinal tract, through the patient's pharynx, and into said trachea.

29. The system of claim 14, wherein said at least one electrode is positionable and fixable on the epicardium of the patient's heart, and wherein said at least one electrode and said at least one lead passable through a wall of said upper gastrointestinal tract.

30. The system of claim 29, further comprising at least one cannula adaptable for passage of said at least one electrical lead through said wall of said upper gastrointestinal tract.

31. A method for implanting a cardiac stimulation system in a patient in need thereof comprising:
implanting in the patient a controller housing comprising a pulse generator; and
positioning at least one electrode, which is carried by at least one electrical lead, at a first selected position within a patient's upper gastrointestinal tract, wherein said at least one electrical lead is attached to said pulse generator.

32. The method of claim 31, further comprising fixing said at least one electrode to epithelial tissue at or about said first selected position.

33. The method of claim 31, wherein said first selected position comprises the esophagus or the stomach of said upper gastrointestinal tract.

34. The method of claim 31, wherein said controller housing is implanted within said upper gastrointestinal tract.

35. The method of claim 34, further comprising positioning at least a second electrode, which is carried by at least a second electrical lead in electrical communication with said pulse generator, at a second selected position within the trachea, the bronchus, the bronchioles, or a branch thereof, of the patient's airway.

36. The method of claim 35, wherein positioning said second electrode further comprises passing said second electrode and said second electrical lead from said upper gastrointestinal tract, through the patient's pharynx, into said trachea, and to said second selected position.

37. The method of claim 35, wherein positioning said second electrode further comprises:
penetrating a wall of said upper gastrointestinal tract to form a first aperture;
penetrating a wall of said trachea or bronchus to form a second aperture; and
passing said second electrode and said second electrical lead from said upper gastrointestinal tract, through said first and second apertures, and to said second selected position.

38. The method of claim 37, further comprising implanting at least one cannula in said first aperture, said second aperture, or a combination thereof, through which said second electrical lead passes.

39. The method of claim 35, wherein said second electrical lead comprises a gastrointestinal lead portion attachable to said pulse generator and adaptable to electrically communicate with a tissue interface within said upper gastrointestinal tract, and an airway lead portion carrying said second electrode and adaptable to electrically communicate with said tissue interface within said trachea or bronchus, wherein positioning said second electrode further comprises guiding said airway lead portion orally through said trachea to said second selected position.

40. The method of claim 31, wherein said controller housing is implanted at a subcutaneous location within the patient.

41. The method of claim 40, further comprising:
forming a subcutaneous tunnel at least from said controller housing implantation site to said upper gastrointestinal tract;
penetrating said upper gastrointestinal tract to form an aperture; and
passing said at least one electrical lead through said aperture.

42. The method of claim 40, wherein positioning said at least one electrode comprises guiding said at least one electrode through said subcutaneous tunnel, through said aperture formed in said upper gastrointestinal tract, and to said first selected position.

43. The method of claim 40, wherein positioning said at least one electrode further comprises guiding said at least one electrode orally into said upper gastrointestinal tract to said first selected position, and further comprising passing the end of said at least one electrical lead opposite said electrode from within said upper gastrointestinal tract, through said aperture formed in said upper gastrointestinal tract, and attaching said at least one electrical lead to said pulse generator.

44. The method of claim 40, wherein said at least one electrical lead comprises a subcutaneous lead portion attachable to said pulse generator and adaptable to electrically communicate with a tissue interface external to said upper gastrointestinal tract, and a gastrointestinal lead portion carrying said at least one electrode and adaptable to electrically communicate with said tissue interface within said upper gastrointestinal tract, wherein positioning said at least one electrode further comprises guiding said gastrointestinal lead portion orally into said upper gastrointestinal tract to said first selected position, and further comprising attaching said subcutaneous portion of said at least one electrical lead to said pulse generator.

45. The method of claim 44, wherein said tissue interface does not form an aperture in said wall of said upper gastrointestinal tract or said trachea or bronchus.

46. A method for electrically stimulating a heart comprising:
positioning and fixing at least one electrode, which is carried by at least one electrical lead, at a first selected position within a patient's upper gastrointestinal tract; and
delivering an electrical signal to said at least one electrode from a pulse generator implanted within said upper gastrointestinal tract or at a subcutaneous location within the patient.

47. The method of claim 46, wherein said pulse generator is operable to deliver one or more electrical pulses effective in cardiac pacing, cardiac defibrillation, cardioversion, cardiac resynchronization therapy, or a combination thereof.

48. The method of claim 46, wherein said pulse generator is implanted at said subcutaneous location, wherein said at least one electrical lead comprises a subcutaneous lead portion attached to said pulse generator and adaptable to wirelessly electrically communicate with a tissue interface external to said upper gastrointestinal tract, and a gastrointestinal lead portion carrying said at least one electrode and adaptable to wirelessly electrically communicate with said tissue interface within said upper gastrointestinal tract, and wherein delivering said electrical signal causes wireless electrical communication from said pulse generator, between said subcutaneous lead portion and said airway lead portion at said tissue interface, to said at least one electrode.

49. The method of claim 46, further comprising positioning and fixing at least a second electrode, which is carried by at least a second electrical lead, at a selected second position within the trachea, the bronchus, the bronchioles, or a branch thereof, of the patient's airway, wherein delivering said electrical signal comprises delivering an electrical signal to said first electrode, said second electrode, or a combination thereof.

50. An endoesophageal cardiac device comprising:
a controller housing proportioned for receipt at a selected housing position within a patient's esophagus, and proportioned to substantially permit fluid and solid flow through said esophagus about said selected position; and
electrical circuitry operable to cause the transmission of at least one electrical signal to at least one electrode.

51. The device of claim 50, further comprising at least one electrical lead attachable to said controller housing, which carries said at least one electrode, said at least one electrode implantable at a selected electrode position in the patient's upper gastrointestinal tract.

52. The device of claim 50, further comprising at least a second electrode, carried by at least a second lead attachable to said controller housing, said second electrode implantable at a selected electrode position within the trachea, the bronchus, the bronchioles, or a branch thereof, of the patient's airway.

53. The device of claim 50, wherein said controller housing further comprises an anchor for fixing said controller housing at said selected controller position.

54. The device of claim 50, wherein said at least one electrical signal comprises one or more electrical pulses effective in cardiac pacing, cardiac defibrillation, cardioversion, cardiac resynchronization therapy, or a combination thereof.

55. A method for implanting a cardiac stimulation system in a patient in need thereof comprising:
delivering a controller housing comprising a pulse generator through the patient's upper gastrointestinal tract;
penetrating a wall of the esophagus of said upper gastrointestinal tract and forming an aperture therein;
passing said controller housing through said aperture;
fixing said controller housing to an external surface of said wall of said esophagus;
delivering at least one electrode, which is carried by at least one electrical lead, through said upper gastrointestinal tract;
passing said at least one electrode and said at least one electrical lead through said aperture; and
fixing said at least one electrode to the epicardium of the patient's heart, wherein said at least one electrical lead is attached to said pulse generator.

56. A method for electrically stimulating the upper gastrointestinal digestive tract of a patient comprising:
positioning and fixing at least one electrode, which is carried by at least one electrical lead, at a selected position within in said upper digestive tract in proximity to at least part of the smooth muscle or the nerves of said upper digestive tract;
delivering an electrical signal to said at least one electrode from a pulse generator implanted within said upper gastrointestinal tract or at a subcutaneous location within the patient.
